(19) United States
(12) Patent Application Publication
(10) Pub. No.: US 2015/0250827 A1
(43) Pub. Date: Sep. 10, 2015

(54) BIO-MIMETIC ULTRATHIN HYDROGEL COATINGS FOR PANCREATIC ISLET TRANSPLANTATION

(71) Applicant: THE UAB RESEARCH FOUNDATION, Birmingham, AL (US)

(72) Inventors: Eugenia P. Kharlampieva, Birmingham, AL (US); Veronika Kozlovskaya, Birmingham, AL (US); J. Anthony Thompson, Birmingham, AL (US); Wanxing Cui, Trabuco Canyon, CA (US)

(21) Appl. No.: 14/126,943

(22) PCT Filed: Jul. 5, 2012
(86) PCT No.: PCT/US2012/045522

§ 371 Date: Dec. 17, 2013

Related U.S. Application Data
(60) Provisional application No. 61/504,367, filed on Jul. 5, 2011.

(51) Int. Cl. A61K 35/39; A61K 35/15; A61K 9/50

(52) U.S. Cl. CPC A61K 35/39; A61K 9/5026; A61K 35/15

(57) ABSTRACT

The present disclosure provides multifunctional cytoprotective materials applied to coat living cells or aggregates of cells such as, but not limited to, pancreatic islets. The coating utilizes hydrogen-bonded interactions of a natural polyphenol (tannic acid) with poly(N-vinylpyrrolidone) deposited on the cell aggregate surface via non-ionic layer-by-layer assembly. The coating is conformal over the surface of such as mammalian islets. The coated islets maintain their viability and cell functionality for at least 96 hours in vitro. The coating demonstrates immunomodulatory cytoprotective properties suppressing pro-inflammatory cytokine synthesis in stimulated bone marrow-derived macrophages and diabetogenic BDC-2.5 T cells. The coating material combines high chemical stability under physiologically relevant conditions with capability of suppressing cytokine synthesis, crucial parameters for prolonged islet integrity, viability, and function in vivo.

### CROSS-REFERENCE TO RELATED APPLICATIONS

This application claims priority to U.S. Provisional Patent Application Ser. No. 61/504,367 entitled “BIO-MIMETIC ULTRATHIN HYDROGEL COATINGS FOR PANCREATIC ISLET TRANSPLANTATION” and filed Jul. 5, 2011, the entirety of which is hereby incorporated by reference.

### STATEMENT ON FUNDING PROVIDED BY THE U.S. GOVERNMENT

This invention was made with government support under NIH Grant Nos.: P30EB011319 and P60DK79636 awarded by the U.S. National Institutes of Health of the United States government. The government has certain rights in the invention.

### SEQUENCE LISTING

The present disclosure includes a sequence listing incorporated herein by reference in its entirety.

### FIELD OF THE DISCLOSURE

The present disclosure is generally related to coatings applied to cells or aggregates of cells by hydrogen bonding to the cell surface. The present disclosure further relates to coated cells or aggregates of cells wherein the viability and functionality of the cells are retained.

### BACKGROUND

Type 1 diabetes (T1 D) is a chronic autoimmune disease representing a major health care problem worldwide. To date, the only reliable strategy to achieve in vivo minute-to-minute glucose control for preventing the devastating complications associated with T1 D is transplantation of insulin-producing cells. Pancreatic islet transplantation (PIT) has been validated as a potentially effective treatment for T1 D. However, there are several limitations to PIT, including: (a) limited availability of donor islets; (b) intraportal islet infusion and associated complications; and (c) the requirement for chronic immunosuppression, resulting in significant side effects (including islet cytotoxicity).

Though pancreatic islet transplantation has emerged as a promising treatment for diabetes, its clinical application however, remains limited due to serious side effects of immunosuppressive therapy necessary to prevent host rejection of transplanted islets (Ricordi & Strom (2004) Nat. Rev. Immunol. 4: 259). Islets secrete a variety of hormones, most importantly insulin, which is responsible for the regulation of blood glucose levels. It is estimated that about 500 thousand to 1 million islets would be needed to treat a patient with type I diabetes (Robertson, R. P.; (2000) New Engl. J. Med. 343: 289-290). With an average islet diameter of 150 μm, this translates into a volume of 1-2 cm³, a considerably smaller volume than that of a whole pancreas. Therefore, the only islets transplantation should result in a far simpler surgical procedure than pancreas transplantation (Meloche, R. M. (2007) World J. Gastroenterol. 13: 6347-6355).

Immunosuppressive therapy is necessary to prevent islet rejection and post-surgery complications. However, lifelong requirement of immunosuppressive drugs has deleterious effects on beta-cell function and on host's ability to fight disease. To protect islets from immune-mediated destruction camouflaging the islet surfaces necessary for immuno-isolation and immunoprotection. Two major approaches have been introduced to prevent immunogenic reactions on the islet surfaces: microencapsulation of the cells and cell surface modification (Chandy et al., (1999) Artif Organs 23: 894-903; Abalovich et al., (2001) Transplant Proc. 33: 1977-1979; de Vos et al., (2003) Biomaterials 24: 305-312; Panza et al., (2000) Biomaterials 21: 1155-1164; Scott & Murad (1998) Curr. Pharm. Des. 4: 423-438; Opara et al., (2010) J. Investigative Med. 58: 831-837). Microencapsulation of islets by a polylysine-alginate polymer complex has been shown to be the most successful example of the microencapsulation strategy (Chang T. M. S. (1995) Biotechnol. Ann. Rev. 1: 267-295). Several studies revealed an increasing survival of islets in vitro when embedded in a solid matrix. Culturing in collagen I gels obtained from rat tail and fibrin gels have shown promising for prolonging islet survival (Wang & Rosenberg (1999) J. Endocrinol. 163: 181-190; Beattie et al., (2002) Diabetes 51: 3435-3439).

Islet encapsulation can provide a means of culturing and delivering islets for transplantation purposes (Beck et al., (2007) Tissue Eng. 13: 589-599). This technique allows for creation of a semi-permeable environment around a group of islets to provide an immune-protection and to allow mass and oxygen transfer. This method demonstrated a great improvement in the biomaterials used for encapsulation and incorporation of various factors enhancing islet functionality. Immune protection of islets by means of encapsulation is an ambitious approach to avoid life-long immune suppression especially in the case of type I diabetes recipients.

For islet encapsulation, the islets are usually entrapped as islets clusters in high-viscous alginate droplets stabilized with divalent ions of barium or calcium. Certain ratios between alginate building blocks, L-guluronic and M-mannuronic acids, should be maintained to prevent inflammation and fibrotic overgrowth after implantation of alginate-encapsulated islets (Zimmermann et al., (2001) Ann. N. Y. Acad. Sci. 944: 199-215). Along with preventing fibrosis, neovascularization of the transplant can increase the long-term viability and function. Recently it was shown for a glucose sensor that it's functioning is increased significantly if revascularization occurs (Klueh et al., (2005) Biomaterials 26: 1155-1163). However, alginates are known to prevent fibroblast adhesion due to its negative charge, which can be considered as one of the fibrosis causes (Schneider et al., (2001) Biomaterials 22: 1961-1970). Most of the approaches used in microencapsulation method including emulsification, discontinuous gradient density centrifugation selective withdrawal and interfacial polymerization result in thick, 5-50 μm, microcapsules (Wyman et al., (2007) Small 3: 683; Sefton et al., (2000) J. Control. Release 65: 17; Calafiore et al., (2001) Ann. NY Acad. Sci. 875: 219; Cruise et al., (1998) Biotechnol. Bioeng. 57: 655). Such microcapsules do not allow for tuning the molecular weight cutoff (or semi-permeable properties of the capsule wall) to prevent recognition by antibodies, and cytokines cannot be sufficiently excluded either (Cui et al., (2004) Transplant. Proc. 36: 1206-1208). Similarly, recently developed polyethylene glycol hydrogels although demonstrated facile control over porosity but the formed microbeads are large and present a barrier for rapid molecular transport (Weber et al., (2007) Cell Transplant. 16: 1049-1057). Moreover, the capsules with larger diameter than islet itself are also expected to plug larger blood vessels than islets do. It imposes harmful effect on the patient liver. The diameter of encapsulated islets must be much smaller than that currently attained to allow transplantation of the islets into portal veins.

Apparently, new methods for the microencapsulation of islets without increasing the diameter of the implant are required. In this respect, modification of islet surfaces would be a powerful tool that can provide an artificial nurturing environment and preserve islet viability and function (Raymond et al., (2004) Biotechnology 32: 275-291; Wilson & Chaikof (2008) Adv. Drug Delivery Rev. 60: 124-145; Ricordi & Strom (2004) Nat. Rev. Immunol. 4: 259-268)

Current islet modification strategies involve covalent conjugation to islet surfaces. Recently, it has been suggested that polyethylene glycol (PEG) grafted onto collagen matrix of isolated islets could inhibit pathways leading to islet xenograft rejection (Xie et al., (2005) Biomaterials 26: 403-412) by reducing antibody/complement-mediated cytotoxicity. PEG-carrying an activated ester, N-hydroxyl-succinimidyl ester group at one ° end was reacted with an amine group of the membrane proteins or collagen layer on the islet surface (Lee et al., (2007) Biomaterials, 28: 1957-1966). In the case when PEG carries the ester groups at both ends, one can be used to covalently bind PEG to the surface and the other for the immobilization of bioactive substances, such as albumin, to mask the surface antigen (Contreras et al., (2004) Surgery 136: 537-547). Using the bifunctional PEG linker, recombinant thrombomodulin was chemically attached to the surface of living islets in a two-step process with the purpose of creating localized anti-inflammatory environment and reducing islet thrombogenicity (Stabler et al., (2007). Bioconjugate Chem. 18: 1713-1715). Various amphiphilic polymers, such as PEG-conjugated phospholipid and polyvinyl alcohol carrying long alkyl chains were used to modify islet surface through hydrophobic interactions (Teramura et al., (2007) Biomaterials 28: 1957-1966; Teramura & lwata (2009) Transplantation 88: 624-630; Totani et al., (2008) Biomaterials 29: 2878-2883. The technologies are limited to the introduction of specified functional small molecules to cells and might perturb cell physiology (Rabuka et al., (2008) J. Am. Chem. Soc. 130: 5947-5953; Paulick et al., (2007) Proc. Natl. Acad. Sci. U.S.A. 104: 20332-20337). Although covalent immobilization was expected to be stable for chemical degradation and present for a long period because of covalent bonding to membrane proteins, introduced polymers and functional groups disappeared from the cell surface with time (Lee et al., (2007) Biomaterials 28: 1957-1966; Cabric et al., (2007) Diabetes 56: 2008-2015). PEG-lipids gradually disappeared from the cell surface without uptake into the inside of cells. They were dissociated from the cell surface into the medium (Teramura et al., (2008) Biomaterials 29: 1345-1355).

Thus, current islet modification strategies mostly involve either covalent conjugation to islet surfaces, which can interfere with cell function (Lee et al., (2007) Biomaterials 28: 1957-1966; Rabuka & Forstner (2008) J. Am. Chem. Soc. 130: 5947-5953), or imbedding islets within thick hydrogels (beads or pallets) that have limited transport of essential nutrients and require large injection volumes unsuitable for transplantation. Moreover, the current designs do not allow for long-term islet contour visualization, a crucial issue in islet transplantation studies (Teramuru & Iwata (2010) Soft Matter 6: 1081-1091; Staedler et al., (2009) Angew. Chem. Int. Ed. 48: 4359-4362; Wilson et al., (2009) J. Am. Chem. Soc. 131: 18228-18229; Brendel et al., (1999) Int. Islet Transplant Registry 8: 5-18). The latter is explained by the direct conjugation of dye molecules to the islet membranes flowed by fast degradation of the conjugation bonds induced for example, by an enzyme cleavage. Another important issue is the preservation of the islet three dimensional structures which is crucial for islet culturing in vitro. Keeping integrity prevents loss of functionality and viability otherwise observed when islets are subjected to traditional attachment culture compared to suspension culture techniques (Paraskevas et al., (1997) Transplant. Proc. 29: 750-752). Therefore, it is important not only improve culture conditions in suspension culture, but also use the strategies which permit the reestablishment of the ECM support and the islet physiological needs.

Layer-by-layer (LbL) surface technique has been applied to modify islet surfaces. The technique is based on alternating layer-by-layer assembly (deposition) of water soluble polymers on solid templates from aqueous solutions ((Kharlampieva & Sukhishvili, (2006) J. Macromol. Sci., Part C-Polymer Reviews 46: 377; Tang et al., (2006) Curr. Opin. Coll. Interface Sci. 11: 203; Lutkenhaus & Hammond (2007) Soft Matter 3: 804; Jiang & Tsukruk (2006) Adv. Mater. 18: 829; Kozlovskaya et al., (2006) Macromolecules 39: 5569-5572). This way, complete coverage of the islet surface can be secured. By selecting specific polyelectrolytes, a defined cutoff of the coating (Kim et al., (2006) Tissue Eng. 12: 221-233) is possible or also inhibitor binding in order to prevent graft rejection, microphage attacks, or antibody recognition. The nanometer-scale thickness can be advantageous for coating individual islets with the ultrathin layer-by-layer films by providing faster response to stimulation and the possibility to bind factors or protective molecules to the protective ultrathin shell with the later slow triggered release of these molecules Chluba et al., (2001) Biomacromolecules 2: 800-805). The negatively charged cell surface is treated with a cationic polymer solution and the cell surface is further exposed to an anionic polymer solution to form an ionically paired polyion membrane. Human pancreatic islets were encapsulated and it was demonstrated islet function and viability by coating them with poly(allyl amine hydrochloride)/poly(styrene sulfonate) (PAH/PSS) multilayer membrane (Krol et al., (2006) Nano Letters 6: 1933-1939; De Koker et al., (2007) Adv. Funct. Mater. 17: 3754-3763).

Despite the significant promise of the LbL strategy for islet modification, the main drawback of the approach is cytotoxicity of the compounds. Current studies mostly focus on electrostatically-bound systems when positively and negatively charged polymers are used for the LbL assembly. Although an LbL membrane through a polyion complex can be achieved on the islet surface, direct interactions between the cationic polymer and cell surface should be avoided because the cell membrane would be gradually destroyed. Most cationic polymers such as poly(L-lysine) and poly(ethylene imine) are extremely cytotoxic and treated cells are severely damaged. The cytotoxic effect has been observed to be dependent on polycation concentration and exposure time (De Koker et al., (2007) Adv. Funct. Mater. 17: 3754-3763). The overall toxic effect of the polyelectrolytes originates from positive charge of polycations inducing pores within cell membrane causing its damage and, eventually, cell death (Bieber et al., (2002) J. Control. Release 82: 441-454; Godbey et al., (1999) J. Biomed. Mater. Res. 45: 286-275; Germain et al., (2006) Biosens. Bioelectron. 21: 1566-1573; Wilson et al., (2008) Nano Letters 8: 1940-1948; Wilson et al., (2009) J. Am. Chem. Soc. 131: 18228-18229). (PLL/alginate)₂ and (PAH/PSS/PAH) multilayers have been found to possess extreme toxicity for human pancreatic islets resulting in their death after only 15 minutes of exposure (Wilson et al., (2008) Nano Letters 8: 1940-1948; Wilson et al., (2009) J. Am. Chem. Soc. 131: 18228-18229). It has been suggested that the dramatic reduction in the toxicity can be achieved by decreasing the polycation charge density through grafting of PEG chains to primary amines on the PLL backbone to generate PLL-g-PEG cytocompatible copolymers (Wilson et al., (2009) J. Am. Chem. Soc. 131: 18228-18229; Wilson, et al., (2011) J. Am. Chem. Soc., 133: 7054-7064).

In regard to cytocompatibility, hydrogen-bonded layer-by-layer approach presents new opportunities for pancreatic islet coating. The properties of the hydrogen-bonded films offer nanocoatings responsive under biologically and physiologically relevant conditions. In contrast to electrostatically-bound LbL, the hydrogen-bonded assembly is based on non-ionic interactions of uncharged polymers and allows incorporating many biocompatible species such as poly(ethylene oxide) (PEO), known to be resistant to protein and lipid adsorption. Similarly, another uncharged polymer, poly(N-vinylcaprolactam) (PVCL), shows a sharp volume transition in the temperature range useful for biomedical applications (lower critical solution temperature, LOST approximately 37° C.). Furthermore, another hydrogen-bonding capable polymer, non-toxic and biocompatible poly(N-vinylpyrrolidone) (PVPON), has been demonstrated to be a key component for materials with low-fouling properties (Zelikin et al., (2007) Nano 1: 63-69; Kozlovskaya et al., (2009) Soft Matter 5: 4077-4087; Kinnane et al., (2009) Biomacromolecules 10: 2839-2846; Kharlampieva et al., (2009) Adv. Mater. 21: 3053; Kozlovskaya et al., (2010) Soft Matter 6: 3596). However, most of the hydrogen-bonded systems involve polycarboxylic acids and therefore can be assembled only at very acidic conditions with pH less than 3, when the acids are protonated (Kozlovskaya et al., (2009) Soft Matter 5: 4077-4087; Kozlovskaya et al., (2010) Soft Matter 6:3596).

Recently, a protocol has been developed for nanoscale hydrogel-like coatings based on hydrogen-bonded interactions of natural polyphenols (tannic acid) with poly(N-vinylpyrrolidone) (Kozlovskaya et al., (2010) Soft Matter DOI: 10.1039/B927369G; Shutava et al., (2009) Nano 3: 1877-1885. Importantly, the system can be fabricated at neutral and slightly basic pHs, crucial for coating under physiological conditions. Tannic acid (TA) has been gearing a strong interest as a molecule with the biological activities including antioxidant, antimicrobial, anti-carcinogenic, anti-mutagenic and antibacterial properties, which is capable of assembling with PVPON and PEO into hydrogen-bonded multilayers of high pH stability (Riedl & Hagerman (2001) J. Agric. & Food. Chem. 49: 4917; Lopes et al., (1999) Biochem. Biophys. Acta 1472: 142-152; Erel-Unal & Sukhishvili (2008) Macromolecules 41: 3962-3970; Hushulian et al., (2003) Biochemistry 68: 1006-1011; Takebayashi et al., (2003) Biol. Pharm. Bull. 26: 1368-1370). Moreover, tannic acid possesses the ability to reduce free-radicals and inhibit radical-induced oxidation of adjacent molecules (Shutava et al., (2007) Russian J. Gen. Chem. 77: 1494-1501; Gustayson, K. H., (1954) J. Polym. Sci. 12, 317; Wilson et al., (2008) Nano Lett. 8: 1940-1948). The permeability through the (TA/PVPON)ₙ shells has been found to depend on the molecular weight of the non-ionic counterpart.

### SUMMARY

The present disclosure provides multifunctional cytoprotective materials applied to coat living cells or aggregates of cells such as, but not limited to, pancreatic islets. The coating utilizes hydrogen-bonded interactions of a natural polyphenol (tannic acid) with poly(N-vinylpyrrolidone) deposited on the cell aggregate surface via non-ionic layer-by-layer assembly. The coating is conformal over the surface of such as mammalian islets. The coated islets maintain their viability and cell functionality for at least 96 hours in vitro. The coating demonstrates immunomodulatory cytoprotective properties suppressing pro-inflammatory cytokine synthesis in stimulated bone marrow-derived macrophages and diabetogenic BDC-2.5 T cells. The coating material combines high chemical stability under physiologically relevant conditions with capability of suppressing cytokine synthesis, crucial parameters for prolonged islet integrity, viability, and function in vivo.

Briefly described therefore, one aspect of the present disclosure, therefore, encompasses embodiments of a biocompatible coating disposed on a cell or aggregate of cells, the coating comprising a first polymer layer attached to a cell or aggregate of cells by hydrogen-bonding, and a second polymer layer disposed on the first polymer layer and attached thereto by hydrogen-bonding.

In embodiments of this aspect of the disclosure, the biocompatible coating can comprise a plurality of alternating first and second polymer layers.

In embodiments of this aspect of the disclosure, the biocompatible coating can comprise between about 3 polymer layers and about 10 polymer layers.

In embodiments of this aspect of the disclosure, the first polymer layer can be selected from the group consisting of: poly(N-vinylpyrrolidone) (PVPON), poly(N-vinylcaprolactam), poly(N-isopropyl-acrylamide), and poly(ethylene glycol).

In embodiments of this aspect of the disclosure, the second polymer layer can be a polyphenolic tannin layer.

In embodiments of this aspect of the disclosure, the at least one polymer layer of the coating can further comprise a functional moiety attached thereto, the functional moiety being selected from the group consisting of: a detectable moiety, an immunomodulatory molecule, a growth factor, or a combination thereof.

In embodiments of this aspect of the disclosure, the outermost polymer layer of the coating can comprise a polyphenolic tannin and the biocompatible coating is immunomodulatory when the coated cell or aggregate of cells is delivered to a recipient animal or human subject.

In some embodiments of this aspect of the disclosure, the coating can comprise a plurality of alternating first and second polymer layers, where the first polymer layer can comprise poly(N-vinylpyrrolidone) (PVPON) and the second polymer layer can be a polyphenolic tannin, where either the first polymer layer or the second polymer layer contacts the cell or aggregate of cells and is attached thereto by hydrogen bonding and the outermost polymer layer of the coating is an immunomodulatory polyphenolic tannin layer, and where at least one polymer layer can have a functional moiety attached thereto, the functional moiety being selected from the group consisting of: a detectable moiety, an immunomodulatory molecule, a growth factor, or any combination thereof.

In embodiments of this aspect of the disclosure, the detectable moiety can be a fluorophore, a radioactive moiety, a metal ion, or a detectable peptide or polypeptide.

In embodiments of this aspect of the disclosure, the cell, or aggregate of cells, can be selected from the group consisting of: a bacterial cell, a viral cell, a plant cell, an artificial cell, and an animal cell.

In embodiments of this aspect of the disclosure, the cell, or aggregate of cells, can be isolated from an animal or human tissue or is a cultured cell or an aggregate of cultured cells.

In embodiments of this aspect of the disclosure, the aggregate of cells can comprise a single species of cell or a plurality of cell types.

In embodiments of this aspect of the disclosure, the aggregate of cells can produce a molecule modulating the physiology of an animal in receipt of the aggregate of cells having the coating thereon.

In embodiments of this aspect of the disclosure, the aggregate of cells, can comprise insulin-producing pancreatic β-cells.

In embodiments of this aspect of the disclosure, the aggregate of animal cells is an isolated pancreatic islet dissected from a pancreas or is a cultured pancreatic islet.

Another aspect of the disclosure encompasses embodiments of a coated aggregate of cells, wherein the aggregate is coated with a first polymer layer hydrogen bonded to the cells of the aggregate or to an extracellular polypeptide thereof.

In embodiments of this aspect of the disclosure, the coated aggregate of cells can further comprise a second polymer layer hydrogen bonded to the first polymer layer, and wherein (i) the first polymer layer is poly(N-vinylpyrrolidone), poly(N-vinylcaprolactam), poly(N-isopropyl-acrylamide), or poly(ethylene glycol) and the second polymer layer is a tannic acid, or (ii) the first polymer layer is a tannic acid and the second polymer layer is poly(N-vinylpyrrolidone), poly(N-vinylcaprolactam), poly(N-isopropyl-acrylamide), or poly(ethylene glycol).

In embodiments of this aspect of the disclosure, the coated aggregate of cells can comprise a plurality of alternating first and second polymer layers.

In embodiments of this aspect of the disclosure, the aggregate of animal cells can be an isolated pancreatic islet dissected from a pancreas or is a cultured pancreatic islet, and wherein the coated aggregate of cells is capable of producing insulin when the coated aggregate is transplanted into a recipient human or animal subject.

In embodiments of this aspect of the disclosure, the at least one layer of the coating can further comprise a functional moiety attached thereto, wherein the functional moiety is selected from the group consisting of: a detectable moiety, an immunomodulatory molecule, a growth factor, or any combination thereof.

Another aspect of the disclosure encompasses embodiments of a method of coating an isolated cell or aggregate of cells, the method comprising the steps of: (a) providing an isolated cell or aggregate of cells; (b) contacting the isolated cell or aggregate of cells with a first compound capable of forming hydrogen bonds with the outer surface of the cell or aggregate of cells, thereby forming a first polymer layer on the outer surface of the cell or cells; and (c) contacting the coated isolated cell or aggregate of cells from step (b) with a second solution of a second compound, thereby depositing the second compound on the first polymer layer hydrogen-bonded to the outer surface of the cell or aggregate of cells.

In embodiments of this aspect of the disclosure, either (i) the first compound is poly(N-vinylpyrrolidone), poly(N-vinylcaprolactam), poly(N-isopropyl-acrylamide), poly(ethylene glycol), and the second compound is a tannic acid, or (ii) the first compound is a tannic acid and the second compound is poly(N-vinylpyrrolidone), poly(N-vinylcaprolactam), poly(N-isopropyl-acrylamide), poly(ethylene glycol).

In embodiments of this aspect of the disclosure, the first polymer layer is poly(N-vinylpyrrolidone) hydrogen-bonded to the outer surface of the cell or aggregate of cells, or to an extracellular matrix component of the cell aggregate.

In embodiments of this aspect of the disclosure, the second compound is a polyphenol.

In embodiments of this aspect of the disclosure, the polyphenol can be a tannic acid.

In embodiments of this aspect of the disclosure, the method can further comprise forming a multi-layered coating by alternately repeating steps (b) and (c).

In embodiments of this aspect of the disclosure, the last step repeated is a step (c).

In embodiments of this aspect of the disclosure, the at least one step (b) the first compound further comprises at least one functional moiety attached thereto.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrates (Left panel) the chemical structures of tannic acid (TA) and poly(N-vinyl-pyrrolidone) and (right panel) the islet encapsulation in a hydrogen-bonded (PVPON/TA)ₙ multilayer using the layer-by-layer assembly. The first PVPON layer was deposited on the islet surfaces through hydrogen-bonded interactions between collagen and PVPON followed by assembly of the TA layer. The layers of PVPON and TA were assembled stepwise until the (PVPON/TA)ₙ multilayer was formed.

FIG. 2 is a scheme illustrating the pathway of synthesis of fluorescently labeled poly(N-vinylpyrrolidone) (FITC-PVPON) as a coating component.

FIG. 3 is a series of confocal laser scanning microscopy (CLSM) images of rat islets coated with (PVPON-TA)₃FITC-PVPON.

FIG. 4 is a series of CLSM images of rat islets coated with one layer of FITC-PVPON.

FIG. 5 is a series of CLSM images of rat islets coated with (PVPON-TA)₃FITC-PVPON and tested in 0, 4, and 7 days.

FIGS. 6A and 6B are CLSM images of NHP (FIG. 6A) and rat (FIG. 6B) islets effectively coated with (PVPON-TA)3FITC-PVPON.

FIG. 7 is a schema illustrating the functionalization of the coating with insulin-stimulating peptide GLP-1 using biotinylation approach.

FIG. 8 is a schema illustrating the PEGylation of a coating of a cell or cell aggregate according to the disclosure.

FIG. 9 illustrates the reaction pathway for the synthesis of functionalized (ALEXA® labeled) PVPON.

FIGS. 10A and 10B are digital images showing the results from coating stability studies on non-human islets.

FIG. 11 is a series of confocal laser scanning microscopy (CLSM) images of non-human primate islets, (PVPON-TA)_{6.5}; CMRL-1066; with PVPON-Alexa.

FIG. 12 is a pair of transmission electron microscope images of non-human primate cells non-coated and coated with (PVPON-TA)₄PVPON.

FIGS. 13A and 13B is a series of CLSM images of human pancreatic islets coated with PVPON-Alexa according to the present disclosure, showing coating of rough islet cells.

FIG. 14 is a series of images from a viability of human islets on 4^{th} day after coating with 4.5 layers according to the present disclosure.

FIG. 15 illustrates a graph showing the thickness of a coating according to the disclosure is dependent on hydration levels.

FIG. 16 is a series of confocal microscopy images of rat (a), NHP (b), and human (c) islets coated with (PVPON/TA)₄PVPON multilayer conformal coatings. Fluorescently labeled PVPON* was assembled in the last bilayer of the coating. The scale bars are 100 μm (a), 50 μm (b), and 100 μm (c).

FIG. 17 is a series of confocal microscopy images of control non-coated (a1, a2, a3) and (PVPON/TA)₆PVPON—coated (b1, b2, b3) non-human primate islets. (c1, c2, c3) CLSM images of the coated islets with higher magnification demonstrating the conformal coating. The left and middle images are taken using 405 nm (‘blue’ channel) and 488 nm (‘green’ channel) laser sources and emission was collected through bandpass filters for wavelengths between 410-506 nm and 494-543 nm, respectively. The right panels are their combined images. The scale bars are 50 μm (a3), 40 μm (b3), and 30 μm (c3).

FIG. 18 illustrates a pair of TEM images of non-coated (left) and (PVPON/TA)₄PVPON-coated (right) NHP islets. The arrows point to the edges of the islet. The scale bars are 100 nm in both images.

FIG. 19 illustrates a pair of confocal microscopy images of (PVPON/TA)₄PVPON-coated NHP islets on the day of coating (Day 1) and after being in culture for 7 days (Day 7) in Miami Medium #1A at 25° C.

FIG. 20 is a graph illustrating the viability of non-coated (control) and (PVPON/TA)₄PVPON-coated rat (blank), NHP (dashed), and human (filled) islets assayed on the day of coating (within 6 hours after film deposition).

FIG. 21 is a graph illustrating the glucose-stimulated insulin release from control (non-coated) and (PVPON/TA)₄PVPON-coated rat islets in response to an increase in glucose concentration from 3.3 mM to 16.7 mM after 24 hours (Day 1) and after 72 hours (Day 4) in vitro in Miami Medium 1A at 25° C.

FIG. 22 is a graph illustrating the stimulation index of the islets on Day 1 and Day 4.

FIGS. 23A-23C are graphs illustrating the effect of (PVPON/TA)₄ shells on Th1 pro-inflammatory cytokine synthesis in stimulated bone marrow-derived macrophages and diabetogenic BDC-2.5 T cells. NOD bone marrow-derived macrophages stimulated with 100 ng mL⁻¹ LPS in the presence of (PVPON/TA)₄ shells for 24 hours exhibited a decrease in IL-12p70 synthesis (FIG. 23A). BDC-2.5 T cells (5×10⁵) stimulated with 1 μM BDC-2.5 mimotope in the presence or absence of 10⁸-10³ (PVPON/TA)₄ shells for 96 hours exhibited a decrease in IFN-γ (FIG. 23B), but no change in IL-2 synthesis (FIG. 23C). Results are representative of 3 independent experiments performed in triplicate. ** indicates p<0.05 versus the control group.

FIG. 24 is a schema illustrating the synthesis of PVPON*. PVPON-co-tBOC was synthesized by copolymerization of VPON and tBOC in dioxane at 65° C. Amino-containing PVPON-co-NH₂ was recovered by dialysis in acidic methanol. Fluorescently labeled PVPON* was obtained by reacting PVPON-co-NH₂ with ALEXA FLUOR® 488 succinimidyl ester in DMF.

FIG. 25 is a pair of optical images of rat non-coated (left) and (TA/PVPON)4-coated islets (right) coated in Miami Medium #1 at 25° C. Concentration of PVPON and TA coating solutions was 1 mg/ml and 0.3 mg/ml, respectively. FDA/PI viability assay test demonstrated viable and non-viable cells. The viability test was performed 60 min after islet coating.

FIG. 26 is a series of optical microscope images of human non-coated (left, top and bottom)) and (PVPON/TA)₄PVPON-coated (right, top and bottom) islets with 1 mg/ml PVPON and 0.5 mg/ml TA deposition solutions in Miami Medium #1. The viability of islets was assessed within 15 min of islet coating (top, left and right) and after 4 day of culturing in Miami Medium #1A. FDA/PI viability assay shows viable and red non-viable cells.

FIGS. 27A and 27B illustrate a pair of fluorescence spectra of Miami culture media taken at Ex=405 nm (FIG. 27A) and Ex=488 nm (FIG. 27B). Measurements were performed using 3 ml 4-sided quartz cuvettes at 25° C. The spectrum from Miami medium #1A supplemented with FCS and ciprofloxacin and used for islet culture is labeled as ‘Medium+supplements’, while that from Miami Medium #1 without the supplements (used for islet coating) is labeled as ‘Medium Only.’ A strong fluorescence signal in the case of ‘Medium+supplements’ was due to ciprofloxacin used as an antibiotic supplement.

FIGS. 28A and 28B are graphs that illustrate (FIG. 28A) growth of (PVPON/TA)n multilayer in Miami Medium #1 at 25° C. monitored by spectroscopic ellipsometry (n denotes a number of deposited bilayers). FIG. 28B is a bar graph illustrating the swelling ratio of the multilayer film assembled on silicon wafers with PEI used as a precursor layer to ensure surface attachment at high pH. Thickness of the film in dry state was used as the minimum thickness value (Tmin). After that, dry film (Bar A) was exposed to pH 7.4 (PBS) for 15 min (Bar B) and for 1 hour (Bar C) and thicknesses of the film were measured in situ, and then the film was dried again and its thickness was measured (Bar D). Swelling ratio was calculated as S,=Thickness/T min.

FIG. 29 is a (top) confocal microscopy image of (PVPONfTA)₄ multilayer hollow shells prepared in Miami Medium #1 at 25° C. (bottom) an SEM image of (PVPON/TA)₄ multilayer hollow shells prepared in Miami Medium #1 at 25° C. and dialyzed in de-ionized water before SEM imaging (hollow shells were dried on silicon wafer at room temperature before imaging).

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

Abbreviations

LbL, layer-by-layer; PVPON, poly(N-vinylpyrrolidone; TA, tannic acid; TEM, transmission electron microscope; FITC, fluorescein isothiocyanate;

### DEFINITIONS

The term “cell” as used herein refers to any natural or artificial cell, animal, plant, bacterial, or a viral particle that be viable or dead. Such cells may be isolated from an animal or human subject or tissue thereof, or a cultured cell previously isolated from a subject source. An artificial cell includes, but is not limited to, an artificially engineered entity derived from such as a unicellular microorganism wherein all or some of the genetic material has been replaced.

The term “aggregate” as applied to a “cell” herein refers to a plurality of a cell type or several cell types that may have been dissected (isolated) from a tissue, or have formed a multicellular body upon culturing in vitro. An exemplary aggregate isolated from an animal tissue is a pancreatic islet of Langerhans. Such an islet may include cells other than those identified as β-cells responsive to a stimulus such as glucose and which, in response thereto, synthesize into the surrounding medium insulin. Such an islet may also be formed in such as a liquid medium by culturing isolated pancreatic cells. Other cell aggregates for use in the compositions of the disclosure can include, but are not limited to, non-pancreatic cells that can produce hormones, cytokines, neuropeptides and the like that may be pathologically deficient in an animal or human subject.

The term “coating” as used herein refers to a multilayered coating encapsulating a cell or aggregate of cells. In such a coating or coat of the present disclosure, a first layer or coat comprises a polymer or units thereof that is hydrogen-bonded to the outer cell membrane surface and, while thus bonded does not significantly reduce the viability, physiology, or functioning of the cell type (for example, by retaining responsiveness to glucose in the case of coated pancreatic islets). It is anticipated that any biocompatible monomer or polymer may be used as the first coating material including, but not limited to, poly(N-vinylpyrrolidone), poly(N-vinylcaprolactam), poly(N-isopropyl-acrylamide), poly(ethylene glycol), silk fibroin, collagen, or a nucleic acid, or any combination thereof.

The term “immunomodulatory” or “modulating an immune response” as used herein includes immunostimulatory as well as immunosuppressive effects. Immunomodulation is primarily a qualitative alteration in an overall immune response, although quantitative changes may also occur in conjunction with immunomodulation. An immune response that is immunomodulated according to the present disclosure can be one that is shifted towards a “Th1-type” immune response, as opposed to a “Th2-type” immune response. Th1-type responses are typically considered cellular immune system (e.g., cytotoxic lymphocytes) responses, while Th2-type responses are generally “humoral”, or antibody-based. Th1-type immune responses are normally characterized by “delayed-type hypersensitivity” reactions to an antigen, and can be detected at the biochemical level by increased levels of Th1-associated cytokines such as IFN-γ, IL-2, IL-12, and TNF-β, as well as IFN-α and IL-6, although IL-6 may also be associated with Th2-type responses as well. Th1-type immune responses are generally associated with the production of cytotoxic lymphocytes (CTLs) and low levels or transient production of antibody. Th2-type immune responses are generally associated with higher levels of antibody production, including IgE production, an absence of or minimal CTL production, as well as expression of Th2-associated cytokines such as IL-4. Accordingly, immunomodulation in accordance with the disclosure may be recognized by, for example, an increase in IFN-γ and/or a decrease in IgE production in an individual treated in accordance with the methods of the disclosure as compared to the absence of treatment.

The term “polyphenol” as used herein refers to structural class of natural, synthetic and semi-synthetic organic chemicals characterized by the presence of large multiples of phenol units generally moderately water-soluble compounds, with molecular weight of 500-4000 Da, at least 12 phenolic hydroxyl groups, and 5-7 aromatic rings per 1000 Da, where the limits to these ranges are necessarily somewhat flexible, and include, but are not limited to the tannins.

The term “(PVPON/TA)ₙPVPON” as used herein refers to a multi-layered coating of a cell or plurality of cells according to the present disclosure, the coating comprising “n” layers, each layer being a first layer of poly(N-vinylpyrrolidone) and a layer of a polyphenol (tannic acid), the poly(N-vinylpyrrolidone) layer being proximal to the underlying cell or cells. “n” denotes the number of layers on the multi-layered coating, “n” ranging from at least one to about 10. In one embodiment of the disclosure, “n” is 3. In embodiments where “n” is 1.5, 2.5, 3.5, 4.5, and the like, the 0.5 denotes that the multi-layered coating has an outer layer of poly(N-vinylpyrrolidone) not having a polyphenol (e.g. tannic acid) layer disposed thereon. In such embodiments, the outermost biocompatible layer, not having a polyphenol layer thereon, may be derivatized for the attachment of such as a labeling moiety, or other functional moiety.

The term “functional moiety” as used herein refers to any molecule that may be attached to the outer surface of the outermost layer of the cell or cell aggregate coatings of the disclosure. It is contemplated, but not intended to be limiting, for such moieties to be an imaging moiety (including a fluorescent dye, radiolabel, and the like), an immunomodulatory molecule, a growth factor, or any combination thereof, and the like.

In describing and claiming the disclosed subject matter, the following terminology will be used in accordance with the definitions set forth below.

### Description

The present disclosure encompasses nanoscale coatings and methods of manufacture thereof suitable for coating isolated cells or aggregates of cells. The coatings are attached to the cells by hydrogen-bonding to proteins on the surface of the cells or to extracellular matrix proteins such as collagen. The coatings of the disclosure, using a hydrogen-bonded, layer-by-layer approach, are particularly advantageous for application to cell aggregates useful for transplantation therapy such as individual pancreatic islets of Langerhans by providing islets suitable for transplantation with the possibility of prolonged viability and physiological functioning compared to other methods of preparation of islets. It is considered within the scope of the invention, however, that the coatings of the disclosure may be used for the preparation of any cell type or group of cells that could usefully provide such as hormonal products, neurotransmitters or neuroregulators, physiological modulators and the like that may be down-regulated or pathologically absent from a human or animal. Besides providing insulin for the treatment of diabetic patients, for example, cells coated by the compositions and methods of the disclosure can be used to provide dopamine for the control of the symptoms of Parkinson's disease, and the like.

Accordingly, the disclosure provides embodiments of cytoprotective coatings comprising, through hydrogen-bonded interactions, such as a natural polyphenol (TA) with poly(N-vinylpyrrolidone) (PVPON) deposited on a group of cells via Layer-by-layer (LbL) assembly. This approach allows for the introduction of material, such as the tannic acid, that is able to modulate adaptive immune responses, crucial for cell enhanced viability and prolonged function after transplantation into a recipient animal or human. In contrast to the currently available approaches based on LbL, the compositions and methods of the disclosure (a) involve non-cationic non-toxic compounds; (b) the compounds can be modified before deposition onto islet surfaces. The coatings are conformal over various types of islets including those derived from rat, non-human primate (NHP), and human. It was found that coated islets maintained their viability and β-cell functional capacity for an extended period, at least 96 hours, in vitro. The coatings also demonstrated immunomodulatory effects by suppressing Th1 pro-inflammatory cytokine synthesis, which greatly benefits success in the establishment and maintenance of transplanted islets.

This approach utilizes conformational hydrogel-like coatings based on hydrogen-bonded interactions of natural polyphenols (tannic acid; TA) with poly(N-vinylpyrrolidone) (PVPON). In contrast to the currently available approaches, this protocol is simple, fast (5-10 min), and non-toxic (hydrogen-bonding at neutral pH for protein assembly), involves biocompatible materials with antioxidant properties (e.g., tannic acid contains biologic activity as an antioxidant, antimicrobial, anti-carcinogenic, anti-mutagenic, and antibacterial agent competent to reduce free radical-induced oxidation of adjacent molecules), is comprised of permeable, ultrathin (less than 100 nm) coatings of minimal void volume, allows for coating of individual islets, and is adaptable to direct functionalization by forming complexes with molecules such as imaging agents, catalytic antioxidants (e.g., CA), and/or immunomodulatory molecules (e.g., FasL). Coating thickness can be easily controlled at the nanoscale level by varying the number of polymer levels. Coating porosity/permeability is controlled by the chemistry of the assembled polymers.

Various conditions, including concentrations of PVPON, TA, and islets were used to optimize rapid and efficient coating of islets with (PVPON/TA)ₙPVPON, where n equals the number of bilayers ranging from 3 to 7. Z-stack confocal imaging demonstrated fluorescent staining on the external surface of islets due to the presence of fluorescently-tagged PVPON. Fluorescence persisted on the surface of islets for over a week in vitro. Hydrogel coating of purified rat, non-human primate (NHP), and human, islets had no immediate effect on islet viability and function (as shown by the glucose-stimulated insulin secretion test, GSIS). Over time (7 days) in culture, (PVPON/TA)-coated islets in suspension culture appeared to retain their morphological appearance (less fragmentation/fusion), an observation that appeared more apparent with increasing coating layers.

Thus, the present disclosure provides methods of synthesis of an ultrathin bio-mimetic coating that can be applied to any type of cell including, but not limited to animal cells, bacterial cells, and the like, that allow the compositions of the disclosure to be attached to the underlying cellular substrate by hydrogen bonding. In one particularly useful application of the methods and compositions of the present disclosure, the coatings and methods of the disclosure can also coat aggregates of cells such as but not limited to, isolated or cultured islets of Langerhans for pancreatic islet modification. The coatings can made of polymeric hydrogels and serve as a protective membrane able to prolong islet viability and functioning of such coated cells or aggregates both in vitro and vivo.

Hydrogen-bonded Layer-by-Layer (LbL) technology can be applied to coat living pancreatic islets. The protocol can be used as an effective alternative approach to polyion LbL-based conformal coating of the islet surface under physiological conditions in a rapid and efficient manner without interfering with the function of the cells. The coatings of the disclosure are (a) conformal with uniform coverage over the whole islet surface; (b) stable for at least 7 days; (c) non-toxic; (d) do not affect islet functions; (e) can be easily modified with functional molecules, such as the fluorescent label FITC; and (f) can be applied to islets or other cell aggregates isolated from a range of animal species such as, but not limited to, rat, non-human primate, and human. It is further contemplated the fabricated ultrathin hydrogen-bonded-based islets coated with cytocompatible fluorescently-labeled polymers may be useful to determine and modulate islet long-term stability, integrity, viability, and function in vitro. For example, but not intended to be limiting, coated Lewis rat islets (syngeneic) can be transplanted into the liver or kidney capsules of streptozotocin-induced (50 mg/kg, intraperitoneally) diabetic Lewis rats. Such a system can then allow determination of the synthesis of biocompatible functionalized polymers; using the pool of synthesized polymers fabrication of nanoscale hydrogel-based coatings; comprehensive coating characterization; and optimization of coating properties and functions.

The thickness of the (PVPON-TA) coating used for rat islet surface modification may be varied by keeping the same number of deposited (PVPON-TA) bilayers (for example 3.5 to about 10 layers) with PVPON of 55,000 Da and by an increased number of bilayers from 3.5 to about 10 in (PVPON-TA) coating with PVPON of 1,300,000 Da. Polymer and polyphenol concentrations can be as established in the coating protocol (see above). Thickness of the produced coatings can be assessed by transmission electron microscopy (TEM) of control (uncoated) and coated islets, or stained, embedded into resin, cut into thin slices, placed on the gold TEM grids and analyzed. In parallel, the (PVPON-TA) systems under investigation were produced on silicon flat substrates under conditions of islet coating (media-based solutions) and their final thicknesses checked with spectroscopic ellipsometry and atomic force microscopy (scratch test). The thickness of the shell can be controlled by changing the molecular weight of PVPON or by increasing the strength of association between the polyphenol and a polymer. For instance, TA/PVPON planar LbL films assembled from PVPON with Mw=55 000 Da and with Mw=1 300 000 Da led to an increased bilayer thickness. The respective bilayer thickness values are 1.0±0.1 nm and 2.2±0.2 nm for the shells fabricated on silica cores. This allows varying the bilayer thickness in the coating of the same chemical composition without changing a number of deposited polymer pairs. In contrast, for ionically assembled (PLL-graft-PEG/alginate) LbL shells used for islet coating, it is necessary to change the deposition conditions, e.g., pH or ionic strength, to achieve the similar effect thus undermining the stability of the coating.

The average TA/PVPON bilayer thickness from AFM cross-sections was 4.0±0.2 nm when deposition was done under high salt conditions (0.1 M) which is as twice as high compared to that of the LbL film adsorbed from low ionic strength solutions (1.7±0.2 nm). The surface microroughness of the films increased significantly as a function of bilayer number from about 2.2 to about 3.1 nm. Such an increase in surface microroughness is consistent with steady development of domain morphology that is caused by weak aggregation of hydrogen bonded components.

(PVPON-TA)ₙ coatings of controlled porosity and permeability necessary for regulating access of molecules towards coated islets were produced, the coatings compatible with islets viability and not interfering with islet function. Functionalization of the (PVPON-TA)ₙ coating surface was through PEGylation and biotinylation due to introduced amine groups mimicking islet surface chemistry. To mimic the surface of islets in terms of present amine groups usually used for islet surface engineering, copolymers of PVPON containing amine groups (the functionalization degree varied in the range of 5-20%) (PVPON-co-NH₂) were synthesized. PEGylation of PVPON-co-NH₂ was carried out through grafting of NHS-PEG chains to amine groups on PVPON backbone to produce PVPON-g-PEG. The grafted PEG were unbranched, hydrophilic, discrete-length molecules in the form of methyl-PEGₙ-NHS ester, where the subscript “n” denotes 4, 8, 12, or 24 ethylene glycol units. The N-hydroxysuccinimide (NHS) ester end group is spontaneously reactive with primary amines (—NH₂), providing for efficient PEGylation of amine-containing molecules or surfaces. Then, the (PVPON-TA)₃ coating was produced around islets according to the procedures of the disclosure and a layer of PVPON-g-PEG was deposited on top. For immobilization of immunomodulating, anti-coagulating or apoptotic ligand, grafting of sulfo-NHS-Biotin or sulfo-NHS-LC-Biotin (Pierce Biotechnology, Inc) to PVPON-co-NH₂ was performed to produce functional PVPON-g-Biotin that could be deposited as a last layer onto (PVPON-TA)ₙ-coated islets. The successful grafting of PEG molecules to PVPON was confirmed by gel permeation chromatography.

Bis-succinimide ester-activated PEG molecules can be used for grafting to PVPON-co-NH₂, to visualize the PEG-terminated coating in confocal microscopy. The N-hydroxysuccinimide ester (NHS) groups at one end of the PEG5 or PEG9 spacer react specifically and efficiently with amino groups on PVPON-coNH₂ at pH 7-9 to form stable amide bonds while the ester group at the other end will be fluorescently labeled with Alexa Fluor 488 carboxylic acid hydrazide sodium salt.

Fusion of rat islets during in vitro culturing at 37° C. has been shown to result in islet decreased viability and necrosis due to limited availability of nutrients and hypoxia. The fusion resistance of the islets coated with (PVPON-TA)₃PVPON-gPEG was studied in vitro. The islets coated with the terminal layer containing PEG corona were cultured in vitro (100 islets/1 mL) and their coating-mediated resistance to fusion was studied by checking the islet morphology and separation through optical microscopy at 3, 5, or 7 days of culturing at 37° C.

Along with protection, the LbL approach of the disclosure is suitable for the simultaneous decoration of the thin coating with visualizing modalities and immunomodulating ligands. The LbL approach allows for a precise control of a number of functional groups introduced within the LbL coating through deposition of polymers with a known degree of functionalization. The fact that polymer can be functionalized “off-line” and the target polymer already bearing necessary functional groups is used in the LbL coating of the islet surfaces ensures that no cell function is disturbed due to cell surface chemistry perturbed by modification.

For example, (PVPON-TA)₃PVPON-g-Biotin islet can be coated with the insulinotropic ligand GLP-1 (Elim Biopharmaceuticals) through the intermediate streptavidin “anchors”. GLP-1 immobilized on the islet surfaces has been shown to enhance insulin secretion in response to high glucose levels compared to that of untreated islets. In another example, (PVPON-TA)₃PVPON-g-Biotin islet can be coated with SA-FasL to study the feasibility of the approach combining advantages of the LbL technology with PROTEXD® technology. The former enables a stable conformal ultrathin coating carrying functional groups capable of covalent binding immunomodulating ligands and of adjustable properties (porosity/permeability/thickness) while the latter provides proteins with potent immune activity displaying them on the conformal artificial membrane for immunomodulation.

Alternatively, carboxylic groups can be introduced along the PVPON polymer backbone by synthesizing PVPON-co-COOH through copolymerization of vinylpyrrolidone (VPON) with 3-(tert-butoxycarbonyl)-N-vinyl-2-pyrrolidone. This copolymer will provide carboxylic functionalities for further covalent binding to amine groups on immunomodulating agents such as FasL or GLP-1 without a need for surface biotinylation. Accordingly, PVPON bearing both carboxylic and hydrazide groups can be produced by converting some of carboxylic groups into hydrazide groups through reaction with tert-butyl carbazate followed by acid deprotection to result in PVPON-co-COOH-co-NHNH₂. Deposition of the PVPON-co-COOH-co-NHNH₂ copolymer as the outmost layer will allow for coating modification with amine- and succinymidyl ester-contained functional ligands.

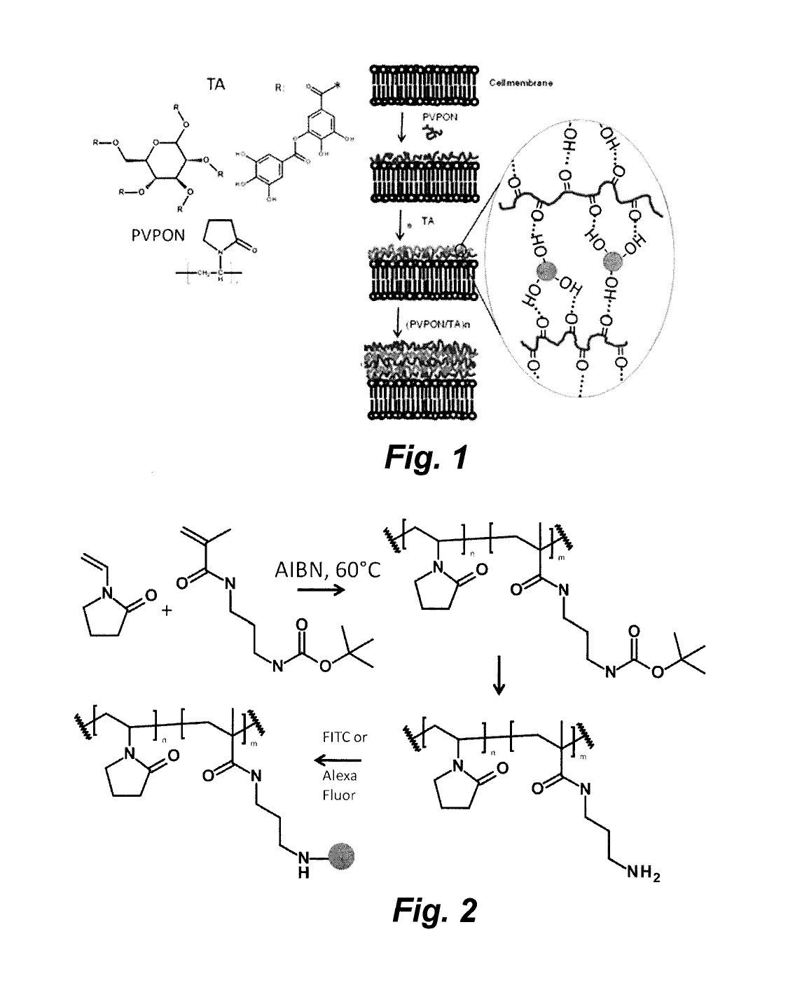

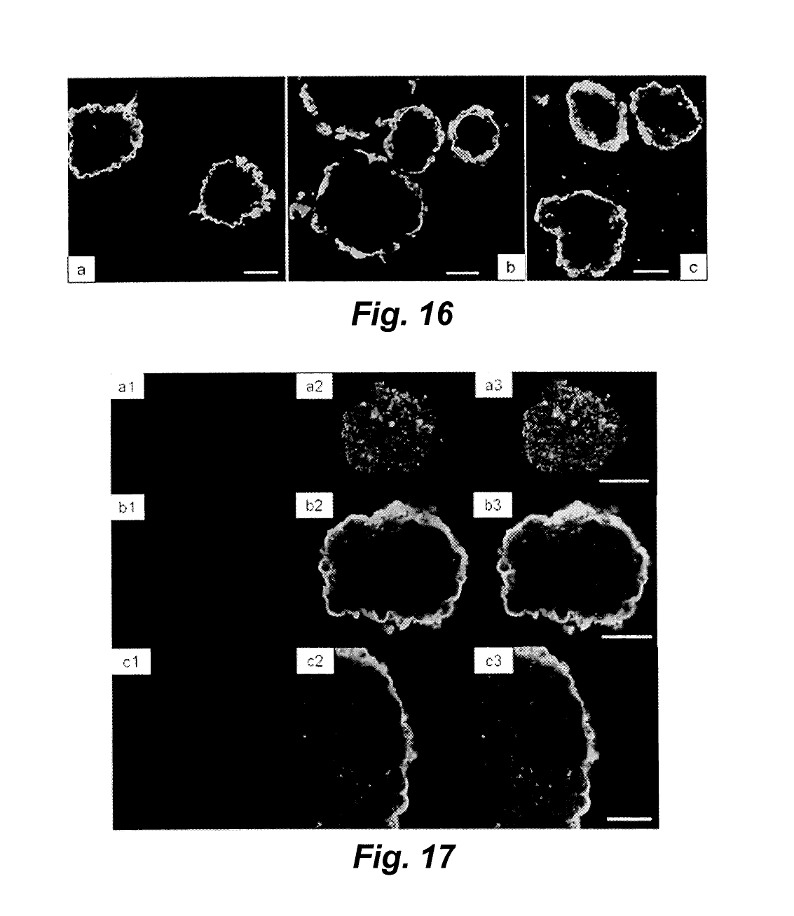

Conformal coating of living islets with a hydrogen-bonded (PVPON/TA)ₙ multilayer film according to the disclosure, where n denotes the number of deposited bilayers, has been established as schematically illustrated in FIG. 1. A layer of non-ionic PVPON can be adsorbed onto the surfaces of islets followed by adsorption of TA. After each deposited layer, islets were collected by centrifugation and washed with media solution before application of the next layer. Alternating polymer deposition onto islets can be continued until the desired number of layers is formed. The results with rat islets demonstrate the uniformity and integrity of a (PVPON/TA)₄PVPON film, as observed with confocal microscopy, as shown in FIG. 16. Most advantageously, the multilayer growth was possible without priming the islet surfaces with such as a polycationic polymer, which has been shown to be detrimental for islet viability (Hong et al., (2006) Bioconjug. Chem. 17: 728).

Direct adsorption of PVPON occurred through non-covalent hydrogen-bonded interactions between the collagen and/or proteins on the islet surfaces and PVPON. Specifically, the pyrrolidone rings in PVPON contain a proton-accepting carbonyl group, while collagen contains carbonyl moieties and N—H groups (as amide bonds), and hydroxyl, side groups that are responsible for the formation of hydrogen bonds. These interactions have been confirmed earlier using FT-IR and DSC techniques and viscosity measurements. The TA layers of the coatings of the disclosure were formed on the PVPON-coated islet surfaces through hydrogen-bonding interactions of the hydroxyl groups on TA and carbonyl groups of PVPON.

For fluorescent visualization of the (PVPON/TA) coating using confocal microscopy, PVPON copolymer were synthesized containing 5% of amine-bearing units using a free radical co-polymerization, as shown in FIG. 2, for example. The PVPON copolymer was been labeled with ALEXA FLUOR® 488 carboxylic acid succinimidyl ester fluorescent dye to produce fluorescently tagged, designated as PVPON*, which was adsorbed in the outermost bilayer. This result demonstrates that while PVPON can be fluorescently labeled through an introduction of reactive functional groups, primary amines, along the PVPON polymer backbone followed by their reaction with the fluorescent dye, PVPON can also be easily modified with various required functionalities (e.g. through PEGylation) and then later used for direct deposition on cell surfaces.

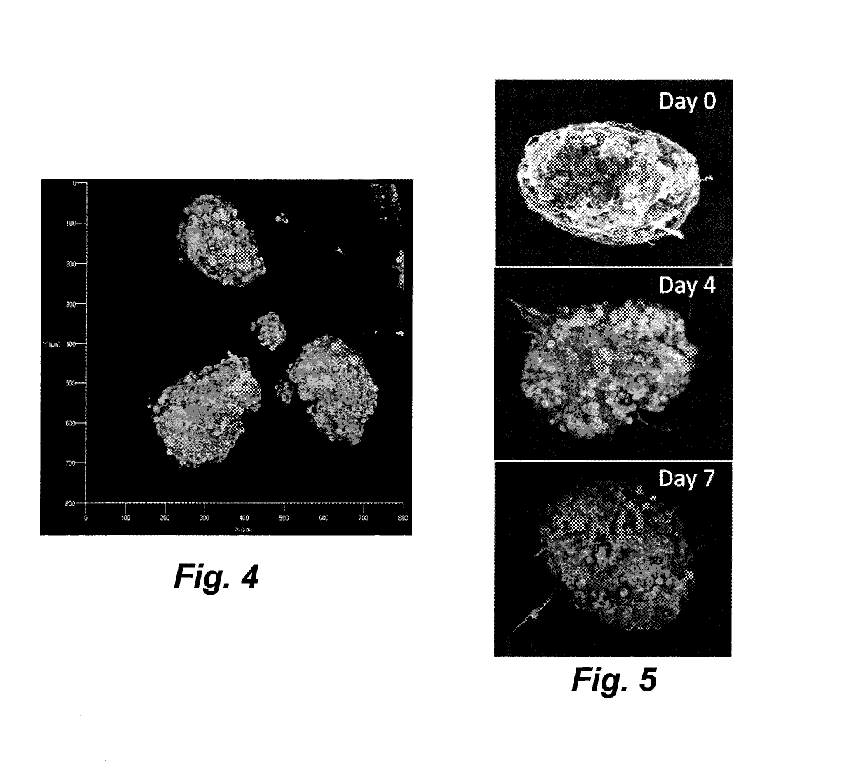

To provide evidence showing that the first PVPON layer was adsorbed to the islet surface, islets were coated with a single PVPON* layer, as shown in FIG. 4. The fluorescent polymer was allowed to adsorb on the islet surfaces for 3 min followed by triple rinsing of the islets with PBS to remove unbound or loosely bound polymer chains from coated-islet suspension and the islets were imaged using confocal microscopy. Evenly distributed fluorescence from the islet surfaces was seen, confirming the deposition of the PVPON* layer that provided the foundation for further (PVPON/TA), multilayer construction.

To establish conditions for homogeneous cytocompatible coating of the islets, a series of islet coating studies were performed. PVPON homopolymer with an average molecular weight of 1,300,000 g/mol and TA with concentrations in the range of about 0.3 to about 1.0 mg/mL for PVPON and from about 0.3 to about 0.5 mg/mL could be used. TA, when deposited as the first layer on the islet surfaces instead of PVPON, also provided the successful hydrogen-bonded (TA/PVPON) multilayer coating. Indeed, polyphenols can bind to proteins present at the cell membranes through formation of multiple hydrogen bonds between phenolic hydroxyl groups of TA and the carbonyl functionalities of the protein peptide bonds.

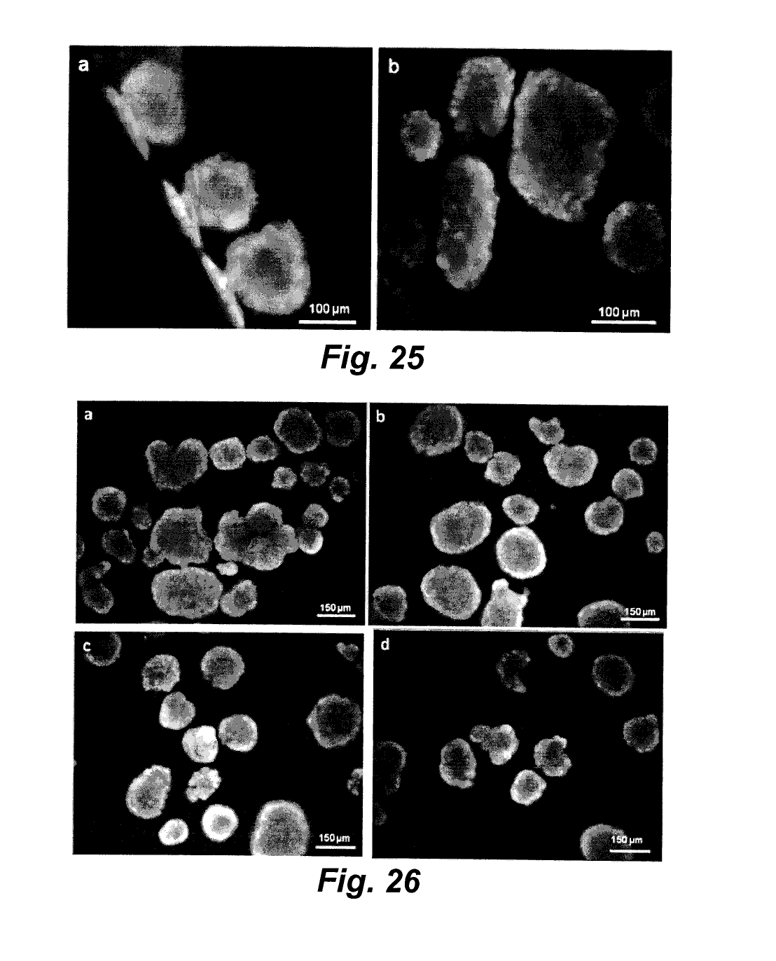

The high binding ability of TA to the cell surface proteins is due to eight TA galloyl groups that promote strong hydrophobic and hydrogen-bonding interactions with cell surface proteins. There was no effect of the TA first layer on cell viability, which indicated that strong interactions between TA and cell membranes were not harmful to the islets, as shown in FIGS. 25 and 26. TA/PVPON coating of encapsulated yeast cells, with TA as the first layer, were previously found to have no effect on the viability (Kozlovskaya et al., (2011) Soft Matter 7: 2364; Carter et al., (2011) Macromol. Biosci. 11, 1244).

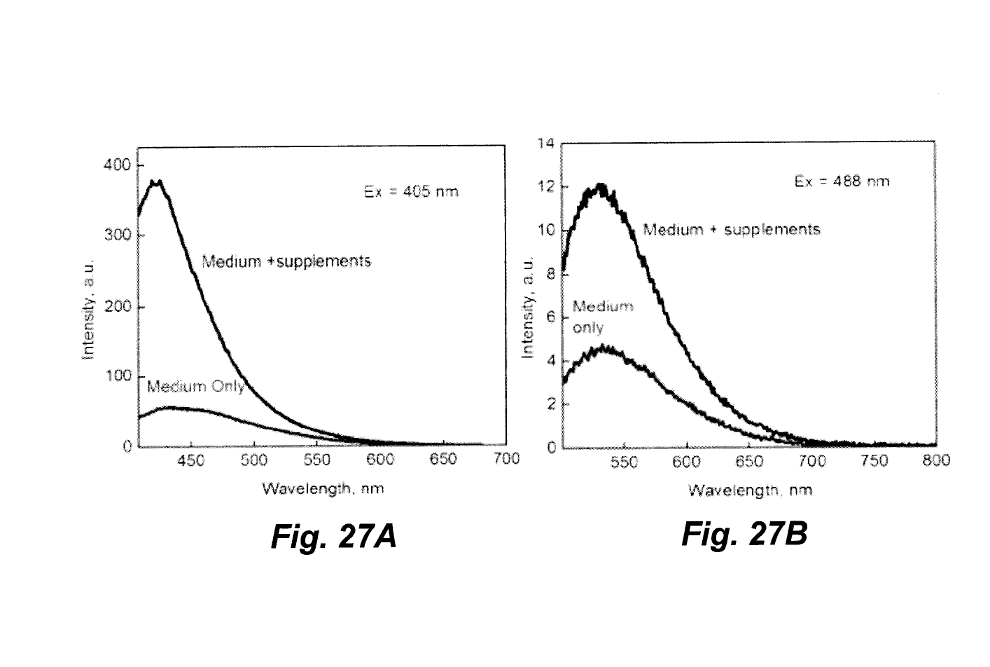

Pancreatic islets from different species can be coated through hydrogen-bonded based LbL of (PVPON/TA). Islet preparation from human cadaveric donors, non-human primates (NHP), and rats were isolated using standard protocols. As shown in FIG. 16 (middle, right), both NHP (FIG. 16, middle) and human (FIG. 16, right) islets can be effectively coated with (PVPON/TA)₄PVPON film. The background fluorescence seen in the images is due to autofluorescence from Miami Medium #1 used as solvent for polymers (FIGS. 27A and 27B).

FIG. 17 shows confocal images of uncoated (control) and (PVPON/TA)₆PVPON-coated NHP islets cultured in Miami Medium #1A. The blue channel images (FIG. 17: a1, b1, c1) in both cases are due to the autofluorescence from the islets themselves, likely from the media amino acid components, while in the green channel the fluorescence from the coating containing labeled PV PON* is seen (FIG. 17: b2, c2). A higher magnification CLSM image of the coated islet in FIG. 17, c2 demonstrates the conformal (PVPON/TA)₆PVPON film on the islet surface.

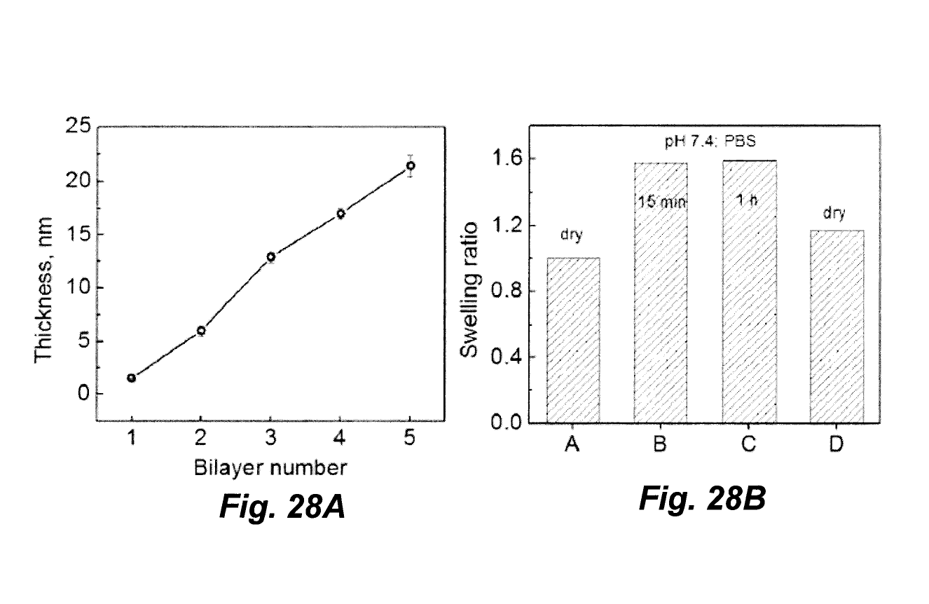

The hydrogen-bonded (PVPON/TA)ₙ film growth was investigated on flat substrates for polymer deposition from Miami Medium#1 by spectroscopic ellipsometry. The ellipsometry data revealed almost a linear growth profile with 4.2±0.4 nm bilayer thickness in dry state (FIGS. 28A and 28B). This value is almost twice as high as that earlier reported for (PVPON/TA) films assembled at pH=7.5 from low salt buffer solutions (Erel-Unal & Sukhishvili (2008) Macromolecules 41: 3962). However, there an increase in the bilayer thicknesses for the (PVPON/TA) film was seen for high salt conditions, which is also the case when the multilayer formation was performed in Miami Medium #1 (ionic strength>0.1 M).

The thickness of the (PVPON/TA)₄PVPON films in the hydrated state was explored using in situ spectroscopic ellipsometry. The data reveals almost 60% thickness increase due to hydration when the films were immersed in PBS solution at pH 7.4 for 1 hour (FIG. 28B).

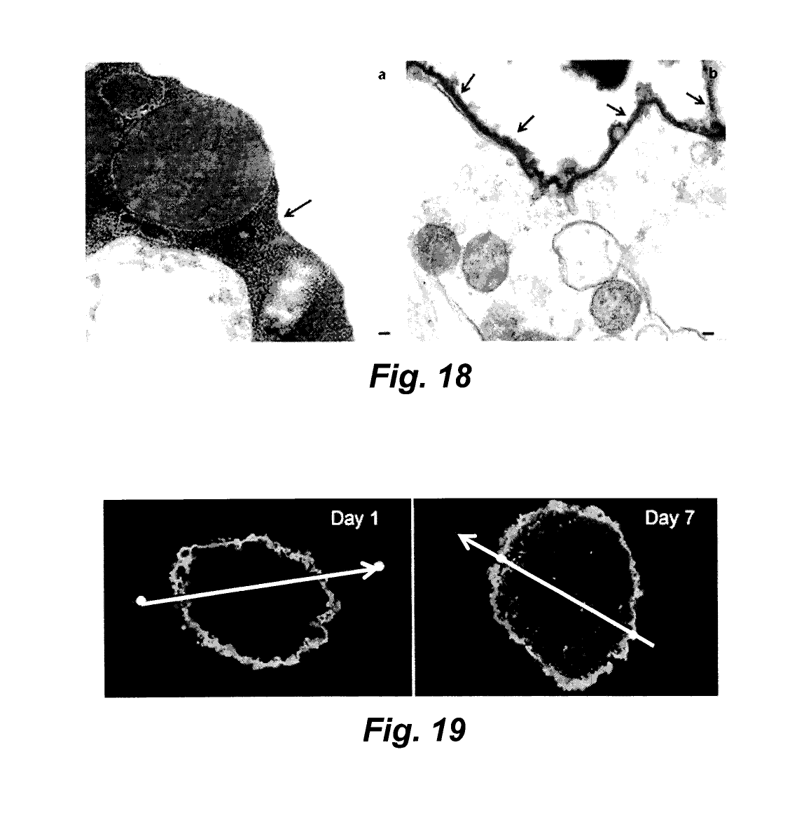

The presence of the (PVPON/TA)₄PVPON coating was further confirmed with transmission electron microscopy (TEM). FIG. 18 compares the outer surfaces of unmodified and modified NHP islets (FIG. 18, left and right, respectively). The TEM images demonstrated the presence of the conformal coating on the surface of coated islets. The (PVPON/TA)₄PVPON film thickness obtained from the TEM analysis resulted in 34±8 nm. This value corresponds to 7 nm per bilayer and correlates well with the ellipsometry data obtained on the planar surfaces (FIG. 28A).

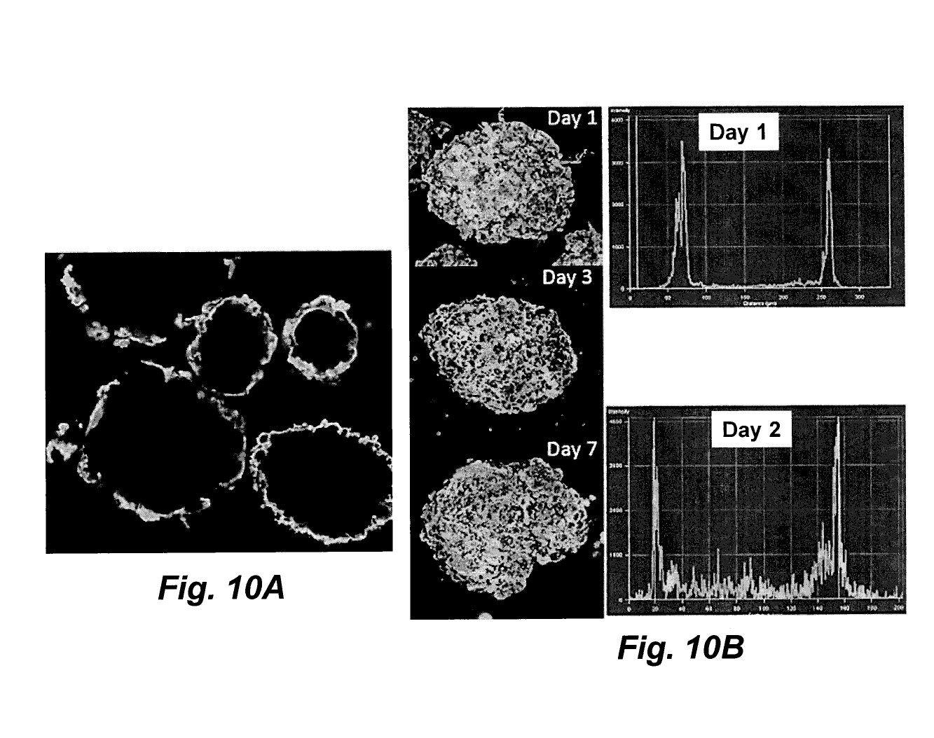

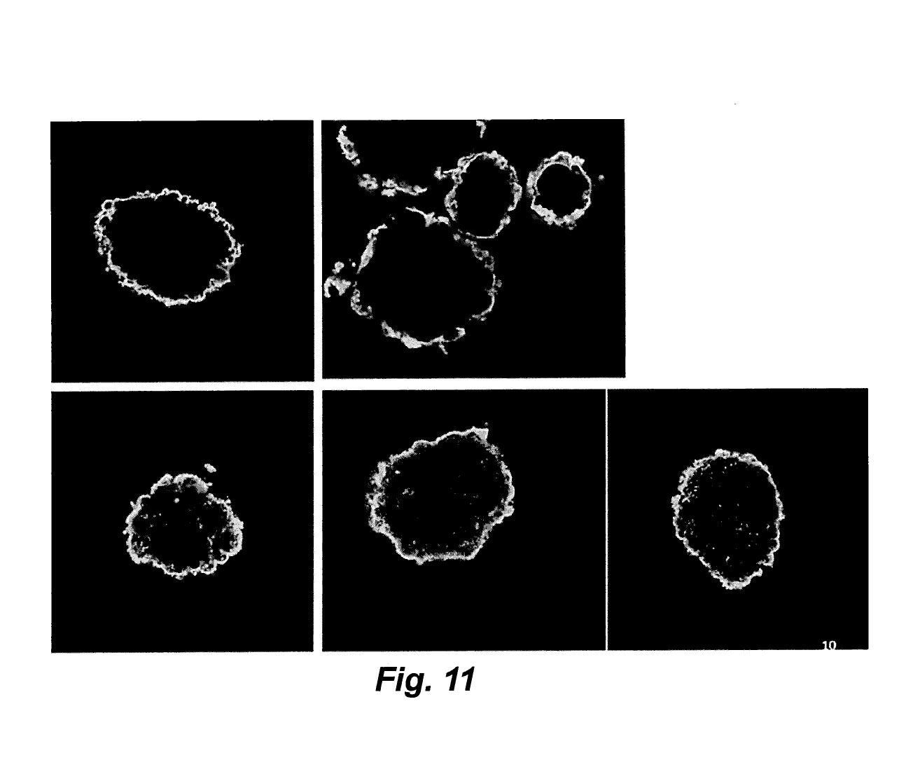

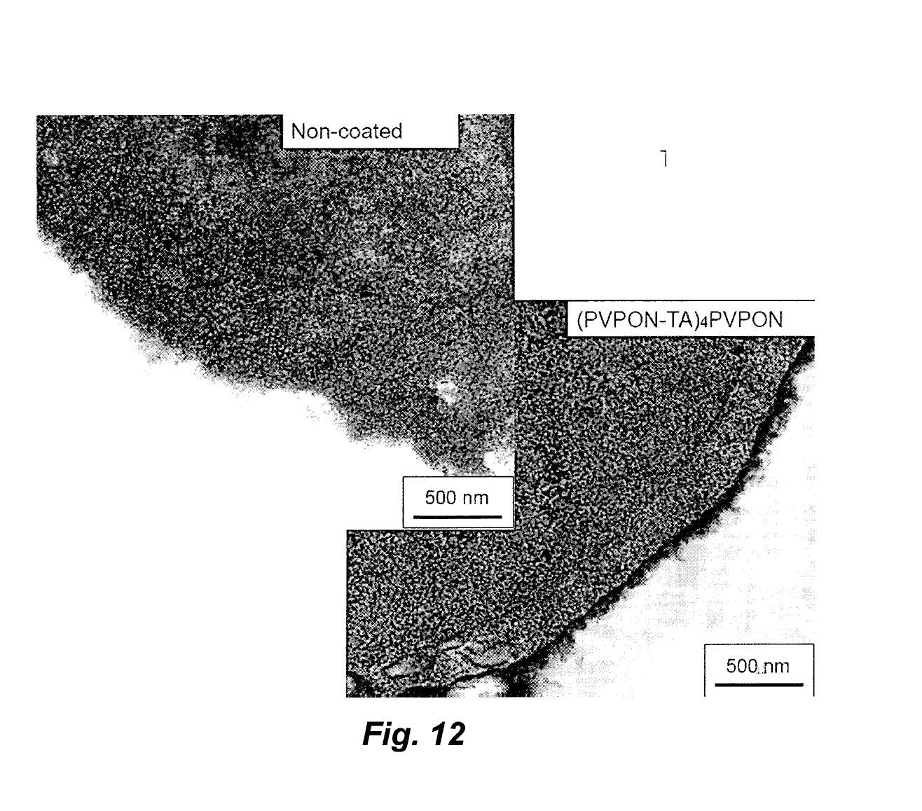

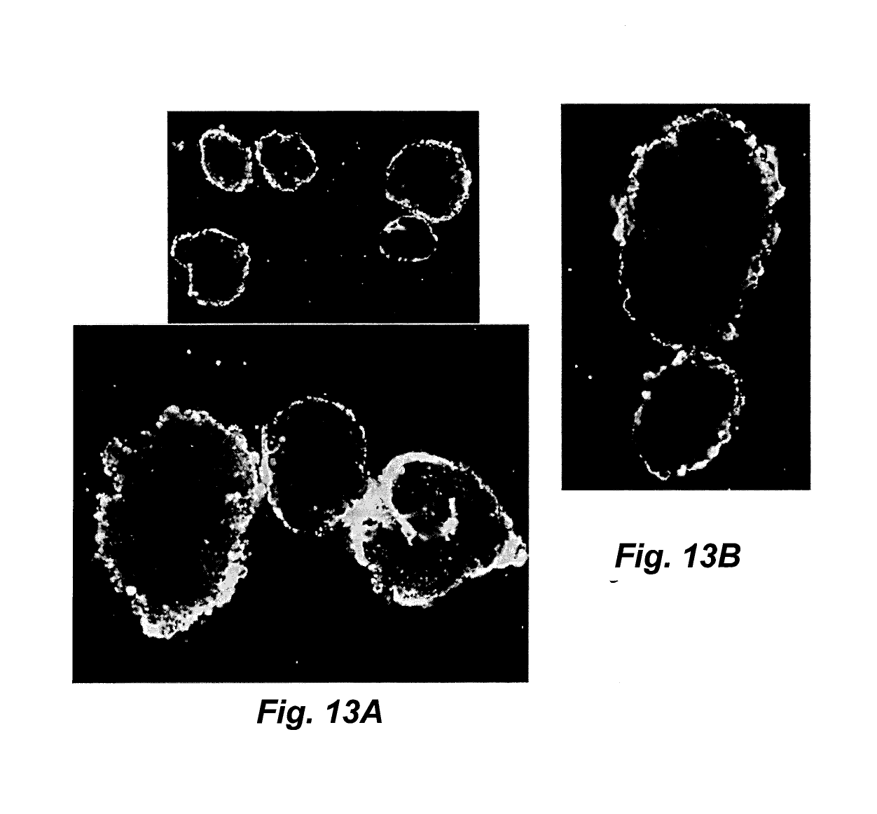

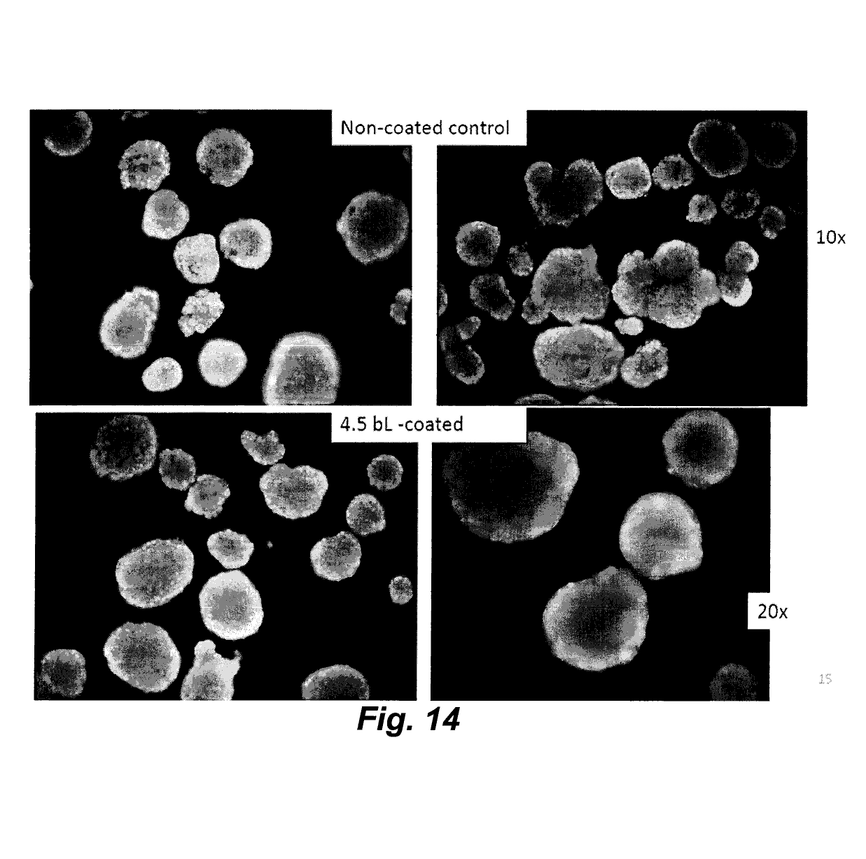

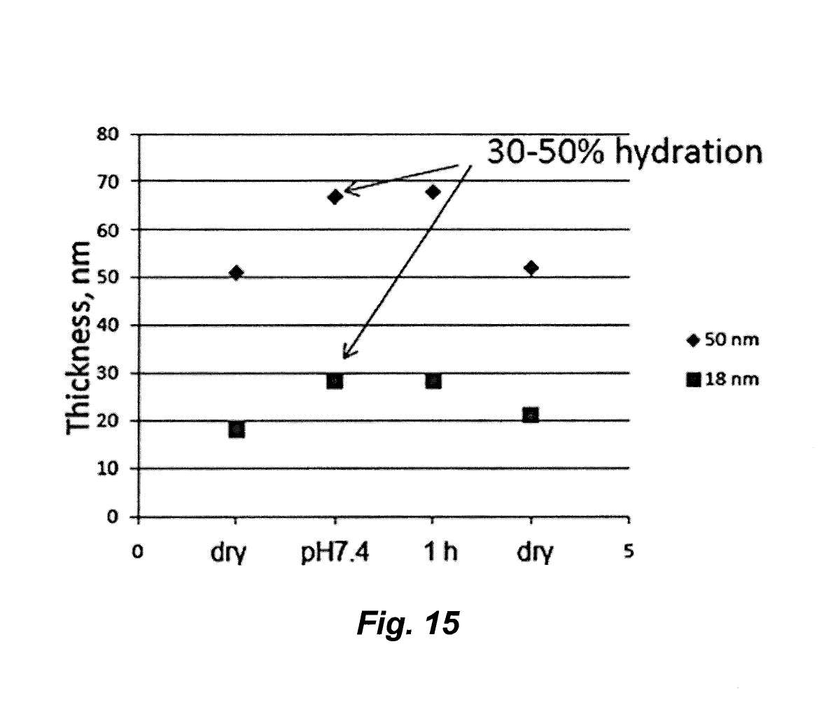

To examine the stability of the film under in vitro conditions, islets were coated with (PVPON/TA)₄PVPON film with a fluorescent layer of PVPON* deposited on top of the coated islets and cultured in vitro in Miami Medium #1A. The islets were harvested at various days and analyzed using confocal microscopy. Confocal microscopy images in FIGS. 10B and 19) revealed conformal coating, demonstrating that the (PVPON/TA)₄PVPON film persisted on the islet surfaces for at least 7 days after initial deposition. No change in the fluorescence intensity of the coated surfaces was found on Day 1 (day of coating) and Day 7, indicating stability of the coating for 7 days (intensity profiles are shown in FIG. 10B, right top and right bottom). A slight increase in intensity observed inside the islet on Day 7 can be explained by the autofluorescence from the Miami Medium #1A, as well as by the autofluorescence related to cells metabolism. Three-dimensional reconstruction of confocal microscope images in FIG. 10B, left) demonstrate evenly distributed fluorescence over the surface of the (PVPON/TA)₄PVPON-coated islets on the day of coating, and after being in culture for 7 days.

Although different types of mammalian islets contain similar types of cells, the islet architectures vary. For instance, insulin-producing β-cells of rodent islets form an inner core of the islet with other secretory cells on the periphery. In human, islets β-cells are interspersed with other cells more on the islet periphery. Due to these morphological differences, it was determined if coatings according to the disclosure could affect the viability of different types of mammalian islets, both rodent islets that are, often used as a research model, and human islets desired to be suitable subjects for transplantation. The viability of rat, NHP and human islets was examined after coating using the FDA/PI viability assay. FDA is cleaved in viable cells and releases a green fluorescent molecule, while a red fluorescent molecule, propidium iodide (PI), can enter only non-viable cells.

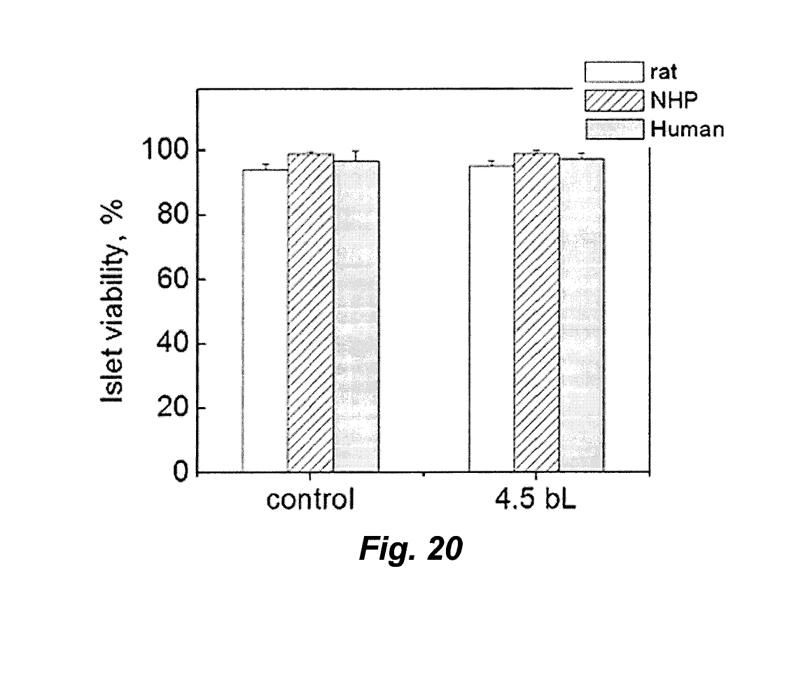

A simple centrifugation method was adopted (3 min deposition time at 2000 rpm) for the islet coating protocol. This method prevented loss of islets during polymer deposition unlike filtration-based methods. FIG. 20 confirms that the viability of islets coated with the (PVPON/TA)₄PVPON protective film was not reduced by the centrifugation method. The viability results for coated islets were statistically indistinguishable from those for uncoated islets regardless of species type. The result also indicates cytocompatibility of the non-ionic PVPON and TA assembled on surfaces of the islets using hydrogen-bonded LbL assembly method. No change in the viability of coated islets was found after 4-day culture in Miami Medium #1, as shown in FIG. 26.

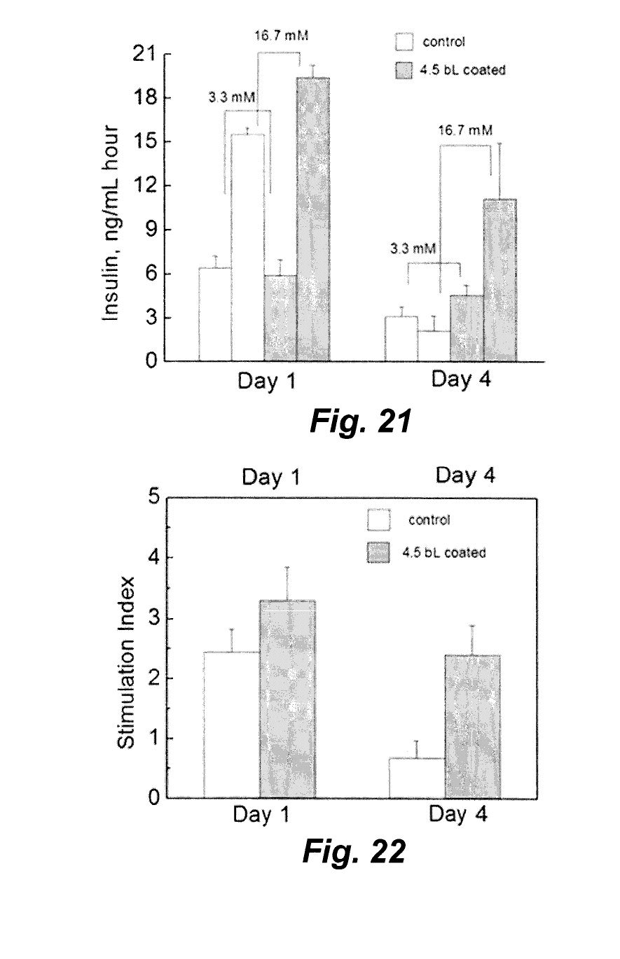

The preservation of appropriate islet functional capacity is critical for successful development of a protective strategy. There was a possibility that the protective coating, while not adversely influencing cell viability, could restrict transport of hormones, e.g., insulin, across the film reducing islet function. To evaluate functionality of the modified islets, therefore, the insulin release in static incubation by non-coated and (PVPON/TA)₄PVPON-coated islets as a function of time, in response to variations in glucose concentration, was determined. The glucose concentration of 3.3 mM refers to a low glucose (basal glucose) level, while 16.7 mM glucose concentration is a high glucose level. As shown in FIG. 21, after 24 hour culture in vitro (Day 1), both control (non-modified) and coated islets exhibited similar insulin secretion level in response to the basal glucose solution, while the insulin response to the high glucose was higher for (PVPON/TA)₄PVPON-coated islets. A significant difference in islet functionality was observed after 96 hours in vitro.

While islets coated with the hydrogen-bonded (PVPON/TA)₄PVPON film preserved their functional capacity, a significant decrease in stimulation index for the untreated islets was seen, as shown in FIG. 22. Such a protective effect of the film can be attributed along with the cytocompatibility of the coating to the ability of the film to retain islet morphological integrity in suspension culture, for example showing less fragmentation and fusion.

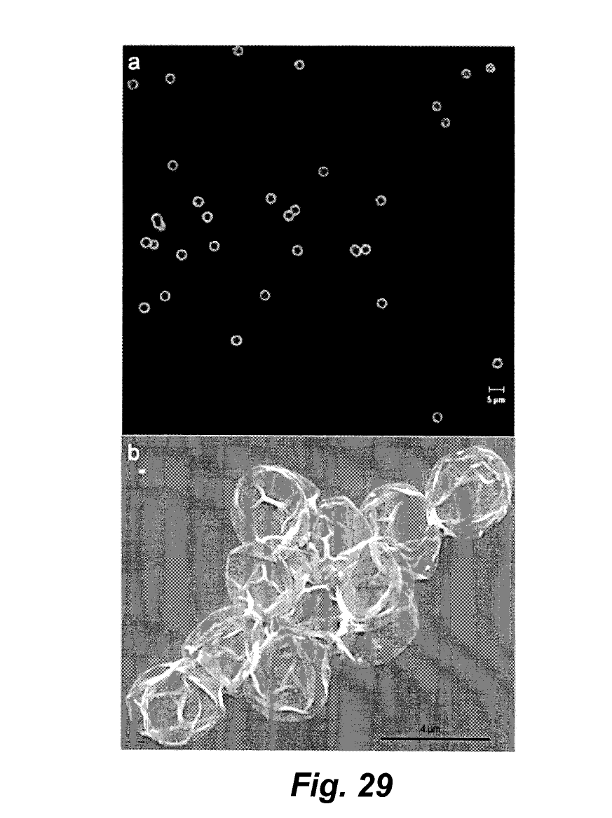

An immunomodulatory effect of the PVPON/TA film based on the idea of a high biological activity of TA, was also examined. The immunomodulatory properties of the coating were studied by investigating the effect of (PVPON/TA)₄ hollow shells on three types of pro-inflammatory cytokines synthesized in stimulated bone marrow-derived macrophages and diabetogenic BDC-2.5 T cells. The shells of (PVPON/TA)₄ were prepared as hollow replicas of 4-μm silica particles coated with (PVPON/TA)₄ multilayers under the conditions used for islet coating. CLSM and SEM images of the shells are presented as FIG. 29.

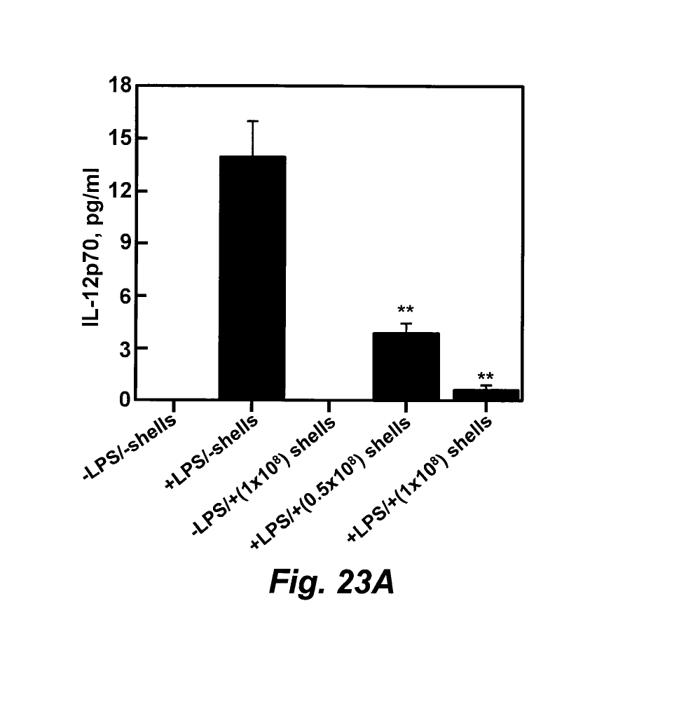

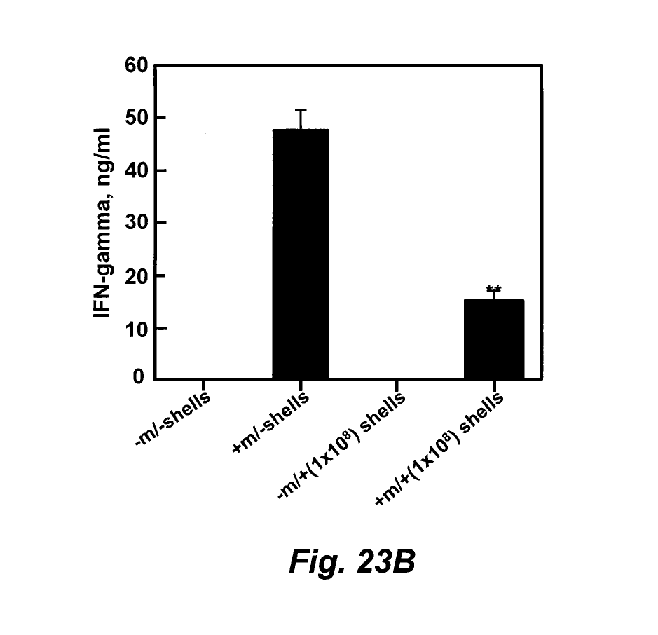

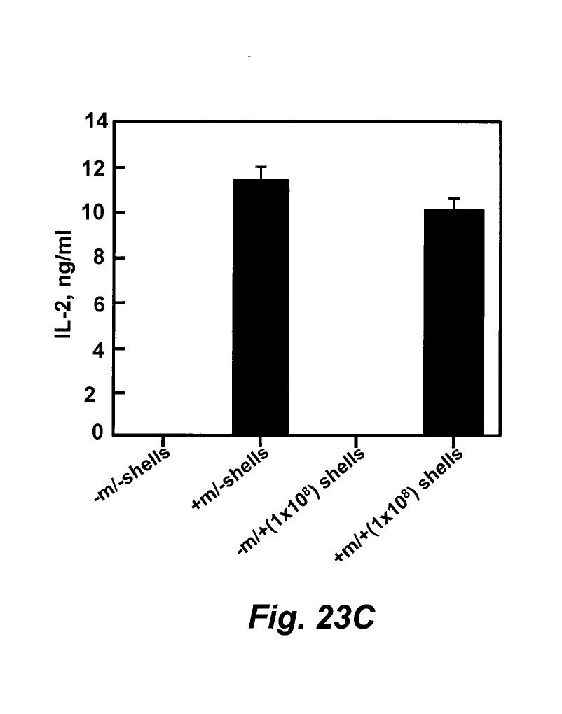

The effect of the shells on synthesis of lymphocyte maturation factor IL-12p70 was tested (FIG. 23A). Thus, 10⁸ (PVPON/TA)₄ shells (counted per 200 μL DMEM) were co-cultured in the presence of bone marrow-derived macrophages stimulated with 100 ng/mL of liposaccharide (LPS). The results were compared with IL-12p70 synthesis in a shell-free environment. The presence and absence of LPS and/or shells in culturing media are denoted as ‘+’ and ‘−’, respectively (FIG. 23A). As seen from FIG. 23A, no IL-12p70 was produced in the absence of LPS in shell-free media. Adding LPS into shell-free media results in synthesis of 14±2 pg/mL of IL-12p70. However, a 10-fold decrease in the IL-12p70 synthesis was observed in the presence of (PVPON/TA)₄ shells. The concentration of synthesized IL-12p70 was found to be inversely proportional to the amount of shells in the culture media. A decrease in the amount of shells from 1×10⁸ to 0.5×10⁸ leads to the increase in IL-12p70 cytokine concentration from 0.6±0.2 to 4.0±0.6 pg/mL (FIG. 23A). Note that 10⁸ shells do not support any cytokine synthesis in LPS-free media, indicating non-immunogenicity of (PVPON/TA).

The (PVPON/TA) shells were then examined for synthesis of interferon-gamma (IFN-γ) and interleukin-2 (IL-2) cytokines, indicative of T cell adaptive immune activation and proliferative capacity, respectively. The syntheses of the cytokines were studied by using diabetogenic BDC-2.5 T cells, as shown in FIGS. 23B and 23C. The BDC-2.5 T cells, also known as autoreactive CD4⁺ T cells, can rapidly transfer Type 1 diabetes into susceptible mice by synthesizing pro-inflammatory T helper type 1 (Th1) cytokines involved in β-cell destruction and macrophage recruitment. To examine the synthesis of IFN-γ and IL-2 cytokines, BDC-2.5 splenocytes were stimulated with their cognate BDC-2.5 mimotope antigenic peptides in the presence or absence of (PVPON/TA)₄ shells (FIGS. 23B and 23C). No IFN-γ and IL-2 cytokines were produced in the mimitope-free media in the absence and presence of shells, which correlates with the results observed in the case of IL-12p70 cytokines in LPS-free media shown in FIG. 23A. Synthesis of mimotope-stimulated IFN-γ was suppressed in the presence of shells, similar to the LPS-stimulated IL-12p70 synthesis. In this case, a three-fold decrease in IFN-γ concentration was observed in the presence of 10⁸ shells, as compared to shell-free media (FIG. 23B). In contrast, no significant change in IL-2 synthesis is found under the same conditions (FIG. 23C).

The data on immunomodulatory properties of the hydrogen-bonded coatings of the disclosure indicate that the shells themselves are not immunogenic, as they do not induce synthesis of the cytokines in the absence of stimulators. Importantly, the shells drastically suppress synthesis of IL-12p70 (about 10-fold) and IFN-γ (about 4-fold) in stimulated macrophages and diabetogenic BDC-2.5 T cells, that recognize chromogranin A, an insulin secretory granule, respectively. IL-12p70 cytokines produce IFN-γ by promoting the formation of T helper type 1 (Th1) effector cells from naïve CD4⁺ T cells (Jacobson et al., (1995) J. Exp. Med. 181: 1755; Murphy et al., (2000) Annu. Rev. Immunol. 18: 451; Robinson & O'Garra, (2002) Immunity 16: 755; Trembleau et al., (1995) J. Exp. Med. 181: 817; G. Trinchieri, (1995) Annu. Rev. Immunol. 13: 251). Since IFN-γ can mediate islet graft rejection, it is contemplated that transplanted islets with (PVPON/TA) coatings according to the disclosure should decrease risk of the islet rejection because of the suppressed syntheses of IL-12p70 and IFN-γ. At the same time, the coating does not affect synthesis of IL-2, thus, is not decreasing the T cell proliferation unlike other approaches for the increase of immunoprotective properties of the coating when immunosuppressive molecules promote T-cell apoptosis. Suppression of the T cell proliferation would be undesirable since a non-specific suppression of the immune system can result in serious chronic side effects including increased risk of infection

One aspect of the present disclosure, therefore, encompasses embodiments of a biocompatible coating disposed on a cell or aggregate of cells, the coating comprising a first polymer layer attached to a cell or aggregate of cells by hydrogen-bonding, and a second polymer layer disposed on the first polymer layer and attached thereto by hydrogen-bonding. In embodiments of this aspect of the disclosure, the biocompatible coating can comprise a plurality of alternating first and second polymer layers.

In embodiments of this aspect of the disclosure, the biocompatible coating can comprise between about 3 polymer layers and about 10 polymer layers.

In embodiments of this aspect of the disclosure, the first polymer layer can be selected from the group consisting of: poly(N-vinylpyrrolidone) (PVPON), poly(N-vinylcaprolactam), poly(N-isopropyl-acrylamide), and poly(ethylene glycol).

In embodiments of this aspect of the disclosure, the second polymer layer can be a polyphenolic tannin layer.

In embodiments of this aspect of the disclosure, the at least one polymer layer of the coating can further comprise a functional moiety attached thereto, the functional moiety being selected from the group consisting of: a detectable moiety, an immunomodulatory molecule, a growth factor, or a combination thereof, and wherein the at least one polymer layer optionally comprises a molecular group suitable for attaching the functional moiety to the polymer layer.

In embodiments of this aspect of the disclosure, the outermost polymer layer of the coating can comprise a polyphenolic tannin and the biocompatible coating is immunosuppressant when the coated cell or aggregate of cells is delivered to a recipient animal or human subject.

In some embodiments of this aspect of the disclosure, the coating can comprise a plurality of alternating first and second polymer layers, where the first polymer layer can comprise poly(N-vinylpyrrolidone) (PVPON) and the second polymer layer can be a polyphenolic tannin, where either the first polymer layer or the second polymer layer contacts the cell or aggregate of cells and is attached thereto by hydrogen bonding and the outermost polymer layer of the coating is an immunomodulatory polyphenolic tannin layer, and where at least one polymer layer can have a functional moiety attached thereto, the functional moiety being selected from the group consisting of: a detectable moiety, an immunosuppressant molecule, a growth factor, or any combination thereof.

In embodiments of this aspect of the disclosure, the detectable moiety can be a fluorophore, a radioactive moiety, a metal ion, or a detectable peptide or polypeptide.

In embodiments of this aspect of the disclosure, the cell, or aggregate of cells, can be selected from the group consisting of: a bacterial cell, a viral cell, a plant cell, an artificial cell, and an animal cell.

In embodiments of this aspect of the disclosure, the cell, or aggregate of cells, can be isolated from an animal or human tissue or is a cultured cell or an aggregate of cultured cells.

In embodiments of this aspect of the disclosure, the aggregate of cells can comprise a single species of cell or a plurality of cell types.

In embodiments of this aspect of the disclosure, the aggregate of cells can produce a molecule modulating the physiology of an animal in receipt of the aggregate of cells having the coating thereon.

In embodiments of this aspect of the disclosure, the aggregate of cells, can comprise insulin-producing pancreatic β-cells.

In embodiments of this aspect of the disclosure, the aggregate of animal cells is an isolated pancreatic islet dissected from a pancreas or is a cultured pancreatic islet.

Another aspect of the disclosure encompasses embodiments of a coated aggregate of cells, wherein the aggregate is coated with a first polymer layer hydrogen bonded to the cells of the aggregate or to an extracellular polypeptide thereof.

In embodiments of this aspect of the disclosure, the coated aggregate of cells can further comprise a second polymer layer hydrogen bonded to the first polymer layer, and wherein (i) the first polymer layer is poly(N-vinylpyrrolidone), poly(N-vinylcaprolactam), poly(N-isopropyl-acrylamide), or poly(ethylene glycol) and the second polymer layer is a tannic acid, or (ii) the first polymer layer is a tannic acid and the second polymer layer is poly(N-vinylpyrrolidone), poly(N-vinylcaprolactam), poly(N-isopropyl-acrylamide), or poly(ethylene glycol).

In embodiments of this aspect of the disclosure, the coated aggregate of cells can comprise a plurality of alternating first and second polymer layers.

In embodiments of this aspect of the disclosure, the aggregate of animal cells can be an isolated pancreatic islet dissected from a pancreas or is a cultured pancreatic islet, and wherein the coated aggregate of cells is capable of producing insulin when the coated aggregate is transplanted into a recipient human or animal subject.

In embodiments of this aspect of the disclosure, the at least one layer of the coating can further comprise a functional moiety attached thereto, wherein the functional moiety is selected from the group consisting of: an detectable moiety, an immunosuppressant molecule, a cytokine, a growth factor, or any combination thereof, and wherein the first or the second polymer layer optionally comprises a molecular group suitable for attaching the functional moiety to the polymer layer.

Another aspect of the disclosure encompasses embodiments of a method of coating an isolated cell or aggregate of cells, the method comprising the steps of: (a) providing an isolated cell or aggregate of cells; (b) contacting the isolated cell or aggregate of cells with a first compound capable of forming hydrogen bonds with the outer surface of the cell or aggregate of cells, thereby forming a first polymer layer on the outer surface of the cell or cells; and (c) contacting the coated isolated cell or aggregate of cells from step (b) with a second solution of a second compound, thereby depositing the second compound on the first polymer layer hydrogen-bonded to the outer surface of the cell or aggregate of cells.

In embodiments of this aspect of the disclosure, either (i) the first compound is poly(N-vinylpyrrolidone), poly(N-vinylcaprolactam), poly(N-isopropyl-acrylamide), poly(ethylene glycol), and the second compound is a tannic acid, or (ii) the first compound is a tannic acid and the second compound is poly(N-vinylpyrrolidone), poly(N-vinylcaprolactam), poly(N-isopropyl-acrylamide), poly(ethylene glycol).

In embodiments of this aspect of the disclosure, the first polymer layer is poly(N-vinylpyrrolidone) hydrogen-bonded to the outer surface of the cell or aggregate of cells, or to an extracellular matrix component of the cell aggregate.

In embodiments of this aspect of the disclosure, the second compound is a polyphenol.

In embodiments of this aspect of the disclosure, the polyphenol can be a tannic acid.

In embodiments of this aspect of the disclosure, the method can further comprise forming a multi-layered coating by alternately repeating steps (b) and (c).

In embodiments of this aspect of the disclosure, the last step repeated is a step (c).

In embodiments of this aspect of the disclosure, the at least one step (b) the first compound further comprises at least one functional moiety attached thereto.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

Tannic acid (TA) (Mw=1700 Da), poly(N-vinylpyrrolidone), (Mw=1,300,000 Da), (PVPON), mono and dibasic sodium phosphate, N-vinylpyrrolidone (VPON). N-(tert-butoxycarbonyl-aminopropyl)methacrylamide (t-BOC), was from Polysciences, Inc.

Fluorescein isothiocyanate fluorescent dye (FITC) and ALEXA FLUOR® 488 succinimidyl ester (Ex/Em=488/520 nm) were purchased from Invitrogen. Ultrapure (Siemens) filtered water with a resistivity of 18.2 MΩ cm was used for preparation of buffered solutions. Initiator, 2, 2′-Azobis(2-methylpropionitrile) (AIBN), was purchased from Sigma-Aldrich and re-crystallized from methanol at approximately 30° C. before use. Miami Medium 1 and 1A were from Mediatech, Inc. BDC-2.5 mimotope peptide (EKAHRPIWARMDAKK (SEQ ID NO.: 1)) was synthesized by Proimmune. Silica microparticles of 4 μm in diameter were purchased from Polysciences Inc.

### Example 2

### Islet Isolation

Islets were isolated from Lewis rats (males, weighing 250-300 g, Harland Laboratories, Indianapolis, Ind., USA), cadaveric human donors, and Rhesus Macaques (NHP) by collagenase digestion. After digestion, islets were isolated by discontinuous density gradient (Ficoll) purification and individually hand-picked under a dissection microscope. All islet samples were cultured at 37° C. in an atmosphere of 95% air and 5% CO₂ in Miami Medium #1A (Mediatech, Indianapolis, Ind., USA) supplemented with 10% FCS and ciprofloxacin (1.0 μg/mL).

### Example 3

### Islet Modification

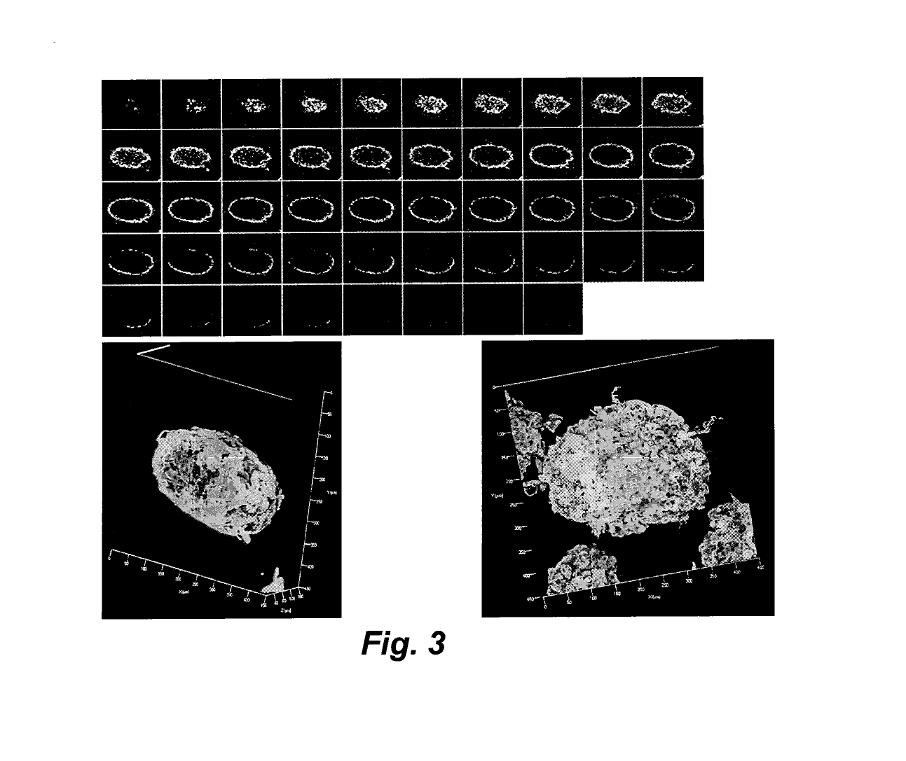

Conformal coating of living Lewis rat islets with hydrogen-bonded (PVPON-TA)ₙ multilayer film has been established and is carried out as schematically illustrated in FIG. 3. Before deposition of (PVPON-TA)n multilayer coating, where n denotes the number of deposited bilayers, islets were pelleted in 1.5 mL Eppendorf centrifuge tubes and washed two times with rinsing solutions of either phosphate buffer (0.01 M in 0.1 M NaCl, pH=7.2) or Miami #1 media. PVPON and TA were sequentially deposited on islet dispersions from aqueous solutions at pH=7.2. Briefly, PVPON was allowed to adsorb first onto islet surfaces from 1 mg/mL solution for 3 min followed by rinsing and deposition of TA layer from 0.3 mg/mL solution for 3 min. After each deposited layer, islets were collected by centrifugation for 2 min at 2000 rpm and washed two times with the rinsing solution. Alternating coating of islet with the polymers was continued until the desired number of layers was achieved. All solutions were filter-sterilized with polystyrene non-pyrogenic membrane systems (0.22 μm pore size) (Corning filter system) before use.

### Example 4

### Glucose Stimulated Insulin Secretion Test

Function of the coated islets was assessed through glucose-stimulated insulin secretion test at 1 and 4 days after coating. All samples were pre-incubated in low glucose Krebs-Ringer bicarbonate buffer (low glucose KRB) (25 mM HEPES, 115 mM NaCl, 24 mM NaHCO₃, 5 mM KCl, 1 mM MgCl₂, 2.5 mM CaCl₂, 0.1% bovine serum albumin, 3 mM D-glucose, pH 7.4) to eliminate residual insulin. After that, each sample was transferred in 1 mL low glucose KRB for 1 hr, followed by incubation in 1 mL high glucose KRB (20 mM D-glucose) for 1 hour. The supernatants were collected and insulin was measured by the ELISA method. Stimulation index (insulin release at high/low glucose concentration) was determined.

### Example 5

### Polymer Synthesis

The protocol for coating Lewis rat islets through layer-by-layer assembly of ultrathin films of hydrogen-bonded synthetic polymer, poly(vinyl pyrrolidone) (PVPON), and natural polyphenol, tannic acid (TA) was developed in both phosphate buffer solution and MIAMI#1 media.

Fluorescently labeled poly(N-vinylpyrrolidone) (FITC-PVPON) for coating visualization in confocal microscopy was synthesized as shown schematically in FIG. 2. PVPON-co-NH₂ copolymer was produced using gradual feeding copolymerization of N-vinylpyrrolidone (VPON) and N-(tert-butoxycarbonyl-aminopropyl)methacrylamide (t-BOC) (Polysciences, Inc.) as described in Kozlovskaya et al., (2008) Soft Matter 4: 1499-1507, incorporated herein by reference in its entirety. Initiator, 2,2′-Azobis(2-methylpropionitrile) (AIBN), was purchased from Sigma-Aldrich and re-crystallized from methanol at approximately 30° C. before use. t-BOC protecting groups were hydrolyzed by treating the copolymer with 1 M HCl in methanol for 100 hours. Solution of the deprotected copolymer was dialyzed against Milli-Q water using a Slide-A-Lyzer Dialysis Cassette (Thermo Scientific) with a molecular weight cutoff of 20,000 Da, and lyophilized (Freeze Dry System). Composition of the resultant amino-containing copolymer prior and/or after hydrolysis of BOC-protective groups was determined using the NMR technique (Bruker DSX 400). The copolymer contained 7% of amine groups. The molecular weight of PVPON-co-NH2 was determined using GPC (Waters). A calibration curve based on linear polystyrene standards was used. The molecular weight of the resultant amino-containing PVPON copolymer was determined to be 79,000 Da.

Amino-containing copolymer PVPON-co-NH₂ was reacted with fluorescein isothiocyanate fluorescent dyes (FITC or Alexa Fluor 488 succinimydil ester) (Ex/Em=488/520 nm) (Invitrogen; in methanol overnight in the dark to produce FITC-PVPON (or Alexa-PVPON) fluorescent polymer. The FITC-PVPON (or Alexa-PVPON) was exhaustively dialyzed against deionized water for several days. The dialysis was completed after no fluorescence was detected in the dialysis water. The dialyzed polymer solution was lyophilized, and FITC-PVPON solution with a required concentration (1 mg/mL) was prepared.

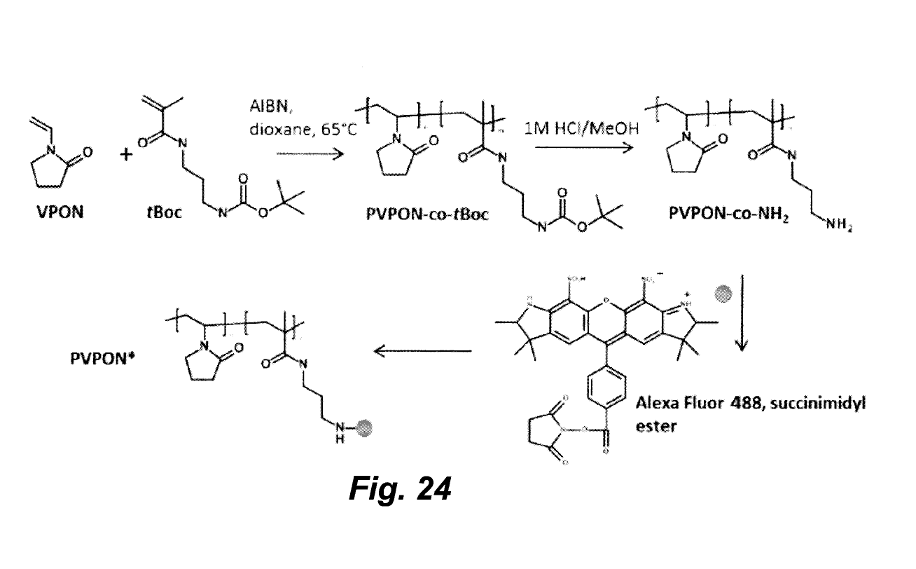

To visualize the (PVPON-TA)ₙ coating, PVPON labeled with FTIC (FITC-PVPON) was used during the deposition of the outermost layer. PVPON can be made fluorescent through introduction of reactive functional groups, primary amines, along the PVPON polymer backbone followed by their reaction with the fluorescent dyes such as FITC or ALEXA FLUOR® 488 carboxylic acid succinymidyl ester, as shown in FIG. 24.

### Example 6

### Confocal Laser Scanning Microscopy (CLSM)

A well of a chambered coverglass (Lab-Tek, Electron Microscopy Sciences) was filled with suspension of coated islets. Confocal images of coated and non-coated islets were obtained with a LSM Zeiss 710 inverted confocal microscope equipped with 10× and 20× objective lenses (Zeiss, Germany). Before imaging, islets were rinsed several times with PBS to reduce auto-fluorescence from the media. For visualization of (PVPON/TA)ₙPVPON* films surrounding the islets, the fluorescence excitation was carried out through a 405 nm (‘blue’ channel) and 488 nm (‘green’ channel) laser sources and emission was collected through bandpass filters for wavelengths between 410-506 nm and 494-543 nm, respectively.

### Example 7

### Evidence of Islet Coating

The preliminary results on rat islets demonstrate the uniformity and integrity of seven-layer fluorescent coatings as observed with confocal microscopy. FIG. 3 illustrates the presence of the (PVPON-TA)₃FITC-PVPON conformal multilayer coating on rat islets. Importantly, the multilayer growth was possible without priming the islet surfaces with a polycationic polymer. Direct adsorption of PVPON was achieved through non-covalent hydrogen-bonded interactions between the collagen on the islet surfaces and polyvinylpyrrolidone. The pyrrolidone rings in PVPON contain a proton accepting carbonyl groups while collagen contains carbonyl moieties and N—H groups (amide bonds) and hydroxyl groups as side groups which are responsible for formation of hydrogen bonds. The possibility of these interactions has been confirmed earlier using FTIR and DSC techniques and by viscosity measurements.

### Example 8

The following TA layer was formed on the PVPON-coated islet surfaces through hydrogen-bonding interactions of the hydroxyl groups on tannic acid and carbonyl groups of PVPON. Tannic acid is able to directly deposit on the islet surface and promote further growth of the hydrogen-bonded (TA-PVPON) multilayer. This is due to the fact that the polyphenol can bind to proteins present at the cell membranes through formation of multiple hydrogen bonds between phenolic hydroxyl groups of tannic acid and the carbonyl functionalities of the peptide linkages of proteins.

### Example 9

A series of islet coating studies were performed using homopolymer PVPON with Mw 1,300,000 Da and TA (Sigma, Belgium) to establish coating conditions that do not compromise the viability and function of the islets. Coating in the range of concentrations of about 0.3 mg/mL to about 1 mg/mL for PVPON and about 0.3 mg/mL to about 0.5 mg/mL for TA sufficed these criteria and provided an effective platform for coating of living cells.

### Example 10

### Coating Stability

To determine the stability of the coating under in vitro culturing conditions, rat islets were coated with (PVPON-TA)₃, with additional layer of FITC-PVPON deposited on top of the coated surfaces of the islets and cultured in vitro in Miami #1 media according to the standard protocol. The islets were harvested various days and analyzed using confocal microscopy. FIG. 5 illustrates that (PVPON-TA)_{3.5} coating was present on the islets for at least 7 days afterwards. The fluorescence intensity decrease observed throughout the observation period is due to gradual photobleaching of FITC moieties on the last PVPON layer. This effect can be minimized when photostable Alexa Fluor 488 is used during preparation of the fluorescent PVPON.

### Example 11

### Transmission Electron Microscopy (TEM)

TEM imaging of control and (PVPON/TA)-coated islets was performed using a FEI-Tecnai T12 Spirit TWIN 20-120 kV electron microscope with AMT digital camera. Sample fixation and staining were done on the 3d day after islet coating with 2% formaldehyde and 1% osmium tetroxide in PBS buffer (0.1 M). Islets were dehydrated with ethanol (50-100%) and suspended in LR White™ resin system (100% ethanol, 1:1 v/v) (Electron Microscopy Sciences).

### Example 12

### Islet Viability Assay

Viability of the non-coated and coated islets was assessed immediately following coating (within 6 hours after film deposition) and after 4 days in vitro (Miami Medium #1A, 25° C.). To evaluate the viability of individual islets coated with the hydrogen-bonded (PVPON/TA) films, control or coated islets were stained with propidium iodide (PI) and fluorescein diacetate (FDA) according to established protocols (Kizilel et al., (2010) Tissue Eng. A 2010, 16, 2217, incorporated herein by reference in its entirety).

An FDA stock solution was prepared by dissolving FDA (10 mg) in acetone (2 mL) and stored at −20° C. For viability test, 10 μL of FDA stock solution were diluted with 990 μL of phosphate buffered saline (PBS). PI (1 mg/mL, Invitrogen) was prepared each time to be used immediately, as 50 μL of solution were diluted with 450 μL of PBS. For viability staining each sample was immersed in a mixture of 2 mL PBS, 10 μL of diluted PI, and 20 μL of diluted FDA. Stained islets were imaged with a Nikon TE 2000-S fluorescence microscope equipped with a Nikon high pressure mercury arc lamp. Fluorescein that de-acetylated from FDA through non-specific esterases in the cytoplasm of living cells was observed under a green fluorescent filter, while PI stained nucleic acids of dead cells under red fluorescent filter. Optical micrographs were converted to binary images comprised of red and green pixels and analyzed using Image J software to quantify the number of pixels corresponding to live (green) and dead (red) cells. Viability was calculated as the percentage of total pixels that were green and an average viability was determined by performing this analysis on 20-25 images of individual islets.

### Example 13

### Rat Versus Primate Islets

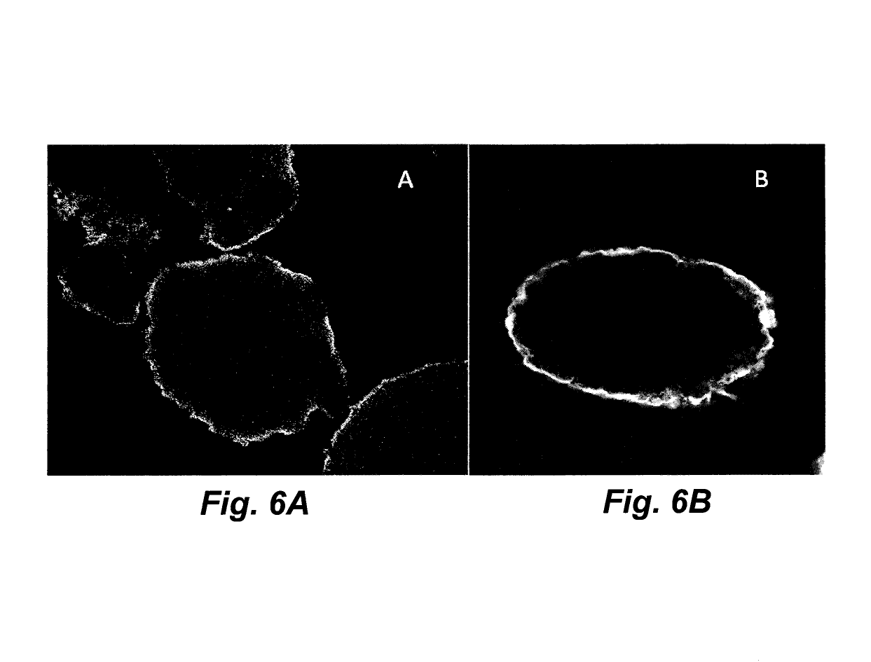

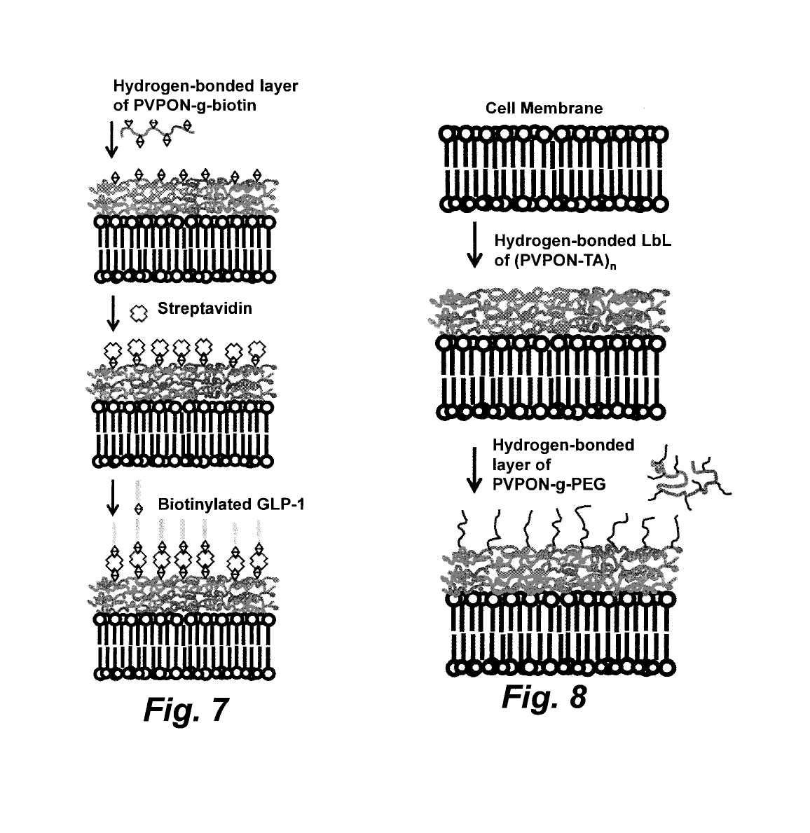

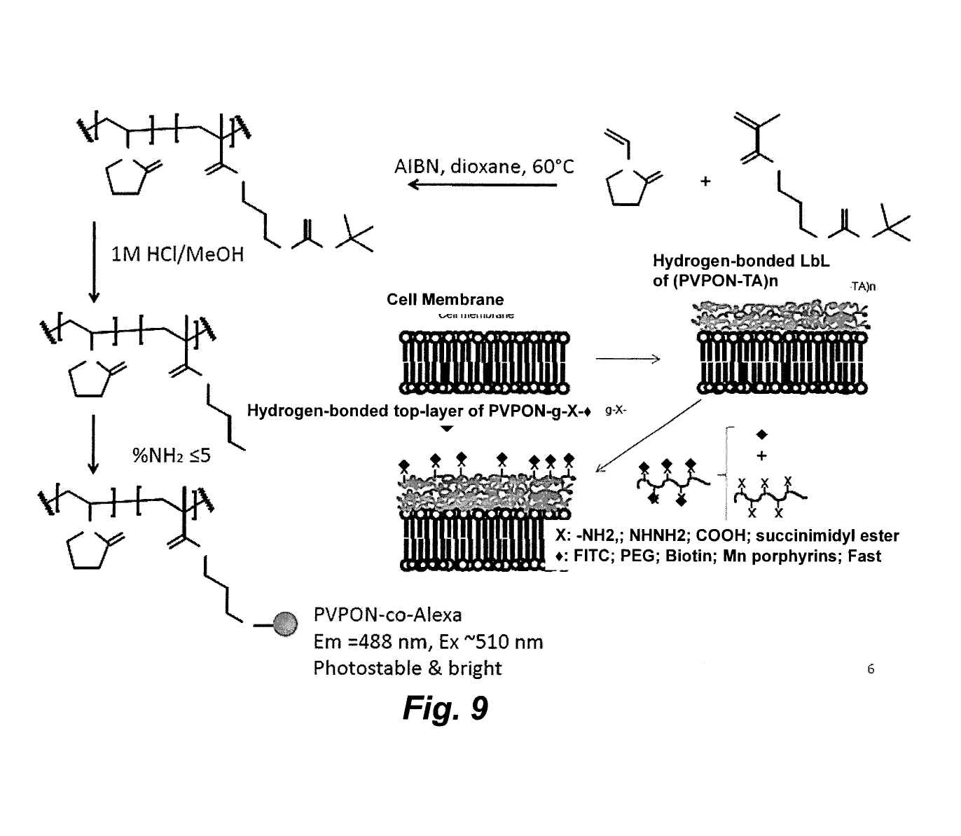

It was tested whether pancreatic islets from different animals can be coated through hydrogen-bonded based LbL of PVPON-TA. Pancreatic islets from non-human primates (NHP) and rats were prepared according to a standard protocol and coated with 3 bilayers of PVPON-TA for 3 min for each layer at room temperature with two intermediated rinsing steps (2 min each) to remove unbound PVPON or TA. Coated islets were then coated with one layer of fluorescent polyvinylpyrrolidone (FITC-PVPON) for 3 min at room temperature, washed twice and observed under confocal microscopy. As shown in FIGS. 6A and 6B, both NHP (A) and rat (B) islets were effectively coated with (PVPON-TA)_{3.5} coating.

### Example 14

The function of the coated islets was assessed through glucose-stimulated insulin secretion test at 1 and 7 days after coating. All samples were pre-incubated in low glucose Krebs-Ringer bicarbonate buffer (low glucose KRB) (25 mM HEPES, 115 mM NaCl, 24 mM NaHCO₃, 5 mM KCl, 1 mM MgCl₂, 2.5 mM CaCl₂, 0.1% bovine serum albumin, 3 mM D-glucose, pH 7.4) to eliminate residual insulin. After that, each sample was transferred in 1 mL low glucose KRB for 1 hour, followed by incubation in 1 mL high glucose KRB (20 mM D-glucose) for 1 hour. The supernatants were collected and insulin was measured by the ELISA method. Stimulation index (insulin release at high/low glucose concentration) was then determined. Coating integrity and stability were tested with confocal laser scanning microscopy (CLSM) at the day of coating and at 3, 5, and 7 days afterwards.

### Example 15

### Preparation of Hollow (PVPON/TA)₄ Shells

Hollow hydrogen-bonded shells (capsules) were prepared by coating 4-μm silica particles with (PVPON/TA)₄ film followed by particle dissolution using the method of the disclosure. Specifically, 1.5 mL of 10% poly(ethylene imine)-coated silica particle suspension was pelleted in a 1.5 mL Eppendorf centrifuge tube and washed twice with Miami Medium #1. TA was allowed to adsorb onto particle surfaces from a 1 mg/mL solution (Miami Medium #1, pH=7.4) for 3 min followed by the deposition of PVPON layer from 0.3 mg/mL solution (Miami Medium #1, pH=7.4) for 3 min. After each deposited layer, particles were centrifuged for 2 min at 2000 rpm and washed two times with the rinsing Miami Medium #1 solution. Alternating coating of particles with the polymers was continued until the desired number of layers was achieved. Silica cores were dissolved in 8% hydrofluoric acid and the hollow capsule solution was dialyzed in de-ionized water at pH=7.4 (adjusted with 0.1 M sodium hydroxide) in the dark for 4 days.

### Example 16

### Primary Recall Assays and Cytokine Measurements by ELISA

NOD.BDC-2.5 splenocyte single cell suspensions (5×10⁵ cells) were seeded in a 96-well flat bottom plate with 0.1 μM or 1 μM BDC-2.5 mimotope in the presence or absence of 10⁸-10³ (PVPON/TA)₄ shells in 200 μL total volume of Dulbecco's Modified Eagle Medium (DMEM) supplemented with 10% heat-inactivated fetal calf serum, 10 mM HEPES buffer, 4 mM L-glutamine, 2× non-essential amino acids, 1 mM sodium pyruvate, 61.5 μM 2-mercaptoethanol, and 100 μg/mL gentamicin (Invitrogen, Carlsbad, Calif.) (complete DMEM). After incubation at 37° C. in a 5% CO₂ humid air chamber for 2, 3, or 4 days, supernatants were collected to examine cytokine synthesis. IFN-γ and IL-2 production was measured using antibody pairs from BD Pharmingen (San Diego, Calif.) as described previously (Tse et al., (2007) Immunol. 178: 908, incorporated herein by reference in its entirety). IL-12p70 was detected with a DuoSet ELISA kit from R&D Systems (R&D Systems, Minneapolis, Minn.). ELISA plates were read on a BioTek Synergy2 microplate reader (BioTek, Winooski, Vt.) and analyzed using Gen5 v.1.10 software (BioTek, Winooski, Vt.).

### Example 17

Isolation and stimulation of mouse bone marrow-derived macrophages. NOD bone marrow-derived macrophages were cultured as described previously (Tse et al., (2002) J. Immunol. 168: 825, incorporated herein by reference in its entirety) and plated on 24-well tissue culture plates at 10⁶ cells per well. Macrophages were stimulated with 100 ng/mL of LPS from E. coli (055:B5) (Sigma Aldrich) in the presence or absence of (PVPON/TA)₄ shells. Supernatants were collected at 24 hours to measure the synthesis of IL-12p70 by ELISA as described (Tse et al., (2007) Immunol. 178: 908, incorporated herein by reference in its entirety).

### Example 18

### Statistical Analysis

Data were analyzed using GraphPad Prism Version 5.0 statistical software. Determination of the difference between mean values for each experimental group was assessed using the 2-tailed Student's t test, with p<0.05 considered significant. All experiments were performed at least three separate times with data obtained in triplicate wells in each experiment.

What is claimed:

1. A biocompatible coating disposed on a cell or aggregate of cells, the coating comprising a first polymer layer attached to a cell or aggregate of cells by hydrogen-bonding, and a second polymer layer disposed on the first polymer layer and attached thereto by hydrogen-bonding.

2. The biocompatible coating of claim 1, comprising a plurality of alternating first and second polymer layers.

3. The biocompatible coating of claim 1, comprising between about 3 polymer layers and about 10 polymer layers.

4. The biocompatible coating of claim 1, wherein the first polymer layer is selected from the group consisting of: poly(N-vinylpyrrolidone) (PVPON), poly(N-vinylcaprolactam), poly(N-isopropyl-acrylamide), and poly(ethylene glycol).

5. The biocompatible coating of claim 1, wherein the second polymer layer is a polyphenolic tannin layer.

6. The biocompatible coating of claim 1, wherein at least one polymer layer of the coating further comprises a functional moiety attached thereto, and wherein the functional moiety is selected from the group consisting of: a detectable moiety, an immunomodulatory molecule, a growth factor, or a combination thereof, and wherein the at least one polymer layer optionally comprises a molecular group suitable for attaching the functional moiety to the polymer layer.

7. The biocompatible coating of claim 1, wherein the outermost polymer layer of the coating comprises a polyphenolic tannin and the biocompatible coating is immunomodulatory when the coated cell or aggregate of cells is delivered to a recipient animal or human subject.

8. The biocompatible coating of claim 1, wherein the coating comprises a plurality of alternating first and second polymer layers, wherein the first polymer layer comprises poly(N-vinylpyrrolidone) (PVPON) and the second polymer layer is a polyphenolic tannin, wherein either the first polymer layer or the second polymer layer contacts the cell or aggregate of cells and is attached thereto by hydrogen bonding and the outermost polymer layer of the coating is an immunosuppressant polyphenolic tannin layer, and wherein at least one polymer layer has a functional moiety attached thereto, the functional moiety being selected from the group consisting of: a detectable moiety, an immunomodulatory molecule, a growth factor, or any combination thereof.

9. The biocompatible coating of claim 8, wherein the detectable moiety is a fluorophore, a radioactive moiety, a metal ion, or a detectable peptide or polypeptide.

10. The biocompatible coating of claim 1, wherein the cell, or aggregate of cells, is selected from the group consisting of: a bacterial cell, a viral cell, a plant cell, an artificial cell, and an animal cell.

11. The biocompatible coating of claim 1, wherein the cell, or aggregate of cells, is isolated from an animal or human tissue or is a cultured cell or an aggregate of cultured cells.

12. The biocompatible coating of claim 11, wherein the aggregate of cells comprises a single species of cell or a plurality of cell types.

13. The biocompatible coating of claim 11, wherein the aggregate of cells produce a molecule modulating the physiology of an animal in receipt of the aggregate of cells having the coating thereon.

14. The biocompatible coating of claim 11, wherein the aggregate of cells, comprises insulin-producing pancreatic β-cells.

15. The biocompatible coating of claim 11, wherein the aggregate of animal cells is an isolated pancreatic islet dissected from a pancreas or is a cultured pancreatic islet.

16. A coated aggregate of cells, wherein the aggregate is coated with a first polymer layer hydrogen bonded to the cells of the aggregate or to an extracellular polypeptide thereof.

17. The coated aggregate of cells of claim 16, further comprising a second polymer layer hydrogen bonded to the first polymer layer, and wherein (i) the first polymer layer is poly(N-vinylpyrrolidone), poly(N-vinylcaprolactam), poly(N-isopropyl-acrylamide), or poly(ethylene glycol) and the second polymer layer is a tannic acid, or (ii) the first polymer layer is a tannic acid and the second polymer layer is poly(N-vinylpyrrolidone), poly(N-vinylcaprolactam), poly(N-isopropyl-acrylamide), or poly(ethylene glycol).

18. The coated aggregate of cells of claim 17, wherein the coated aggregate of cells comprises a plurality of alternating first and second polymer layers.

19. The coated aggregate of cells of claim 13, wherein the aggregate of animal cells is an isolated pancreatic islet dissected from a pancreas or is a cultured pancreatic islet, and wherein the coated aggregate of cells is capable of producing insulin when the coated aggregate is transplanted into a recipient human or animal subject.

20. The coated aggregate of cells of claim 16, wherein at least one layer of the coating further comprises a functional moiety attached thereto, wherein the functional moiety is selected from the group consisting of: an detectable moiety, an immunomodulatory molecule, a growth factor, or any combination thereof, and wherein the first or the second polymer layer optionally comprises a molecular group suitable for attaching the functional moiety to the polymer layer.

21. A method of coating an isolated cell or aggregate of cells, the method comprising the steps of:
(a) providing an isolated cell or aggregate of cells;
(b) contacting the isolated cell or aggregate of cells with a first compound capable of forming hydrogen bonds with the outer surface of the cell or aggregate of cells, thereby forming a first polymer layer on the outer surface of the cell or cells; and
(c) contacting the coated isolated cell or aggregate of cells from step (b) with a second solution of a second compound, thereby depositing the second compound on the first polymer layer hydrogen-bonded to the outer surface of the cell or aggregate of cells.

22. The method of claim 21, wherein either (i) the first compound is poly(N-vinylpyrrolidone), poly(N-vinylcaprolactam), poly(N-isopropyl-acrylamide), poly(ethylene glycol), and the second compound is a tannic acid, or (ii) the first compound is a tannic acid and the second compound is poly(N-vinylpyrrolidone), poly(N-vinylcaprolactam), poly(N-isopropyl-acrylamide), poly(ethylene glycol).

23. The method of claim 21, wherein the first polymer layer is poly(N-vinylpyrrolidone) hydrogen-bonded to the outer surface of the cell or aggregate of cells, or to an extracellular matrix component of the cell aggregate.

24. The method of claim 21, wherein the second compound is a polyphenol.

25. The method of claim 24, wherein the polyphenol is a tannic acid.

26. The method of claim 21, further comprising forming a multi-layered coating by alternately repeating steps (b) and (c).

27. The method of claim 24, wherein the last step repeated is a step (c).

28. The method of 26, wherein in at least one step (b) the first compound further comprises at least one functional moiety attached thereto.

29. The method of 28, wherein the functional moiety is selected from the group consisting of: an imaging moiety, an immunomodulatory molecule, a growth factor, or any combination thereof.

30. The method of claim 29, wherein the imaging moiety is a fluorescent moiety.

31. The method of 21, wherein the cell or aggregate thereof is selected from the group consisting of: a bacterial cell, a viral cell, a plant cell, an artificial cell, and an animal cell, and wherein the cell or aggregate of cells is an isolated cell or aggregate of cells or a cultured cell or aggregate of cells.

32. The method of claim 21, wherein the aggregate of cells is an isolated human or animal pancreatic islet or population of islets.

33. The method of claim 32, wherein the pancreatic islet or population of islets is dissected from a pancreas or comprises cultured pancreatic islet cells.


SEQUENCE LISTING (1 sequence)

SEQ ID NO: 1 - protein, 15 aa, Artificial sequence
  BDC-2.5 mimotope peptide
EKAHRPIWARMDAKK